(19) United States
(12) Patent Application Publication
(10) Pub. No.: US 2024/0131088 A1
(43) Pub. Date: Apr. 25, 2024

(54) METHOD FOR PREPARING A TRANSFORMED, SALMO SALAR INTERFERON GAMMA (IFNG)-PRODUCING LACTOCOCCUS LACTIS BACTERIUM

(71) Applicants: CONSORCIO TECNOLOGICO DE SANIDAD ACUICOLA S.A., Santiago (CL); UNIVERSIDAD DE SANTIAGO DE CHILE, Santiago (CL)

(72) Inventors: Mario Cesar Gerardo TELLO REYES, Santiago (CL); Alvaro Eugenio SANTIBAÑEZ VARGA, Santiago (CL); Mick Philippe PARRA MARDONEZ, Santiago (CL); Diego Enrique PAINE CABRERA, Santiago (CL); Claudia Andrea ZAPATA ROJAS, Santiago (CL); Andrea Del Pilar GARCES FERNANDEZ, Santiago (CL)

(21) Appl. No.: 18/467,280

(22) Filed: Sep. 14, 2023

Related U.S. Application Data
(62) Division of application No. 16/764,356, filed on Jun. 26, 2020.

(30) Foreign Application Priority Data
Nov. 14, 2017 (CL) 2897-2017

(51) Int. Cl. A61K 35/744; A23K 10/18; A23K 50/80; A61K 9/00; A61K 38/21; C12N 1/20; C12N 15/74

(52) U.S. Cl. CPC A61K 35/744; A23K 10/18; A23K 50/80; A61K 9/0053; A61K 38/217; C12N 1/205; C12N 15/746; C12R 2001/46

(57) ABSTRACT

The present invention falls within the technical field of aquaculture, and specifically, the invention relates to a specific solution for preventing and treating bacterial diseases using a Lactococcus lactic lactic acid bacterium transformed to produce an interferon type II (IFN II) immunostimulating cytokine, particularly interferon gamma (IFNg or IFNy). Said transformed bacterium has been deposited in the Chilean Microbial Genetic Resources Collection at INIA with accession number RGM 2416 dated 22 Oct. 2017.

### CROSS-REFERENCES TO RELATED APPLICATIONS

This application is a divisional patent application of US patent application No. 16/764,356, filed on May 14, 2020, which is a national stage entry of PCT/CL2018/050021, filed on Apr. 20, 2018, all of which are hereby incorporated by reference in their entireties.

### AN INCORPORATION BY REFERENCE STATEMENT REGARDING THE MATERIAL IN

An XML file for a “Sequence Listing XML” (see § 1.831(a), submitted via the USPTO patent electronic filing system, identifying
the name of the file: DNA Sequence for CU-76114;
the date of creation of each file: Friday, Dec. 29, 2023
and the size of each file in bytes 36,846 bytes.

### FIELD OF INVENTION

The invention belongs to the field of aquaculture, and in particular discloses a solution for preventing and treating bacterial diseases using a transformed lactic acid bacteria to produce an immunostimulant cytokine type II interferon (IFN II), particularly Interferon gamma (IFNg or IFNy). This transformed bacteria was deposited into INIA's Chilean Collection of Microbial Genetic Resources and has been assigned the accession number RGM 2416, dated Oct. 22 2017. Likewise, the invention relates to an immunostimulant feed for aquatic species that comprises the transformed lactic bacteria with salmonid type II Interferon. Also concerns the preparation of the probiotic feed, the preparation method of the IFNy expressing lactic bacteria i.e., the preparation of a cytokine that stimulates antibacterial immune response and an immunomodulator kit against bacterial infections for aquatic species.

### BACKGROUND OF THE INVENTION

In salmon farming, different natural causes produce fish mortality, being those of infectious origin the most aggravating. Some of the pathogens that have caused biggest losses are bacteria Flavobacterium psycrophilum and Pisciricketsia salmonis. The main strategy to stimulate fish immune response in aquaculture is by using injectable vaccines. However, this type of immunization involves manipulation and causes high stress to fishes. Furthermore, due to fish's short immune memory continuous boosters are needed, which require manipulation, promoting further stress, mortality and reducing the quality of the fillet. Moreover, oral vaccination and immersion strategies have limited effectivity and its use has restrictions.

Together with the current state of available vaccines, diseases of bacterial origin, mainly those caused by P. salmonis, have not been controlled using vaccines which has led to the excessive use of broad spectrum antibiotics, which can impact the environment around fish cages, can increase antibiotic resistance of pathogens and alter fish microbiome. These drawbacks push the need for the creation of new non-invasive methods to stimulate fish immune response. Current research is focused in oral vaccines that can be used as boosters, allowing repetitive immune stimulations without the need of manipulating until harvest.

Interferon are a group of cytokines that were originally discovered due to their antiviral properties (Isaacs and Lindenmann, 1957). In mammals, these cytokines can be classified into three families according to their homology, receptors, structure and function (Pestka et al., 2004). Type I and II interferons are families of multiple genes (17 for type I and 4 for type II) strongly induced by virus, and have an important role controlling viral infections (Lazear et al., 2015, McNab et al., 2015, Teijaro, 2016). Type II interferon or Interferon gamma (IFNy) is an immunoregulatory cytokine coded by one gene, produced mainly by NK cells and activated T cells, although it is also secreted by B cells and antigen-presenting cells (ARC) (Frucht et al. 2001, Kearney et al., 2013, Zimonjic et al., 1995).

Type I interferon from Salmo salar (Atlantic salmon) has shown antiviral properties that induce the expression of several antiviral genes, among these Mx (Ooi et al., 2008, Martin et al., 2007, C. Xu, Evensen and Munang'andu 2015). In vitro and in vivo experiments have shown that type I interferon is effective against pathogens of economic relevance such as IPNV, IHNV, ISAV and SAV (Chang et al., 2014, 2016, Robertsen et al., 2003, Saint-Jean and Perez- Prieto, 2006, Sun et al., 2011, Svingerud et al., 2012, 2013, Xu et al., 2010). Furthermore, type I interferon in Salmo salar also stimulates adaptative immunity, increasing immunostimulation produced by DNA vaccines (Chang et al., 2015).

However, bioactivity assays using recombinant Salmo salar or rainbow trout IFNg have shown that this cytokine is capable of inducing the expression of MHCI and MHCII proteins, which are involved in the processing of antigens and increasing the respiratory burst of phagocytic cells (Martin et al., 2007a, 2007b, 2007c, Zou et al., 2005). Additionally, IFNg has shown antiviral properties in vitro against SAV and IPNV (Sun et al., 2011).

As in human beings, IFNs have the potential to be used in salmon farming as therapeutics or in prophylaxis treatments against virus. However, the use of these cytokines has been unsuccessful in aquaculture, mainly due to lack of an appropriate vehicle, compatible with fish physiology that makes its use effective.

The present invention relates to the development of bacteria to be used as an oral immunostimulant for non-specific stimulation of the innate response against bacterial infections, of either intracellular or extracellular pathogens. The present invention shows that salmon IFN gamma (IFNγ) confers protection against bacterial infections in salmonids by activating genes that increase cellular and humoral immune response of fish. Therefore, expression of the immunostimulant cytokine in the mucus membrane, using acid lactic bacteria as liberation vehicle, confers a protective effect on fish against bacterial infections.

In the last decades, lactic acid bacteria (LAB) have emerged as a feasible vehicle for in situ liberation of cytokines and bioactive peptides within the gastrointestinal tract of mammals (Bahey-El-Din et al., 2010; Steidler et al., 2009). Interferons alpha (Bayat et al., 2014, Ma et al., 2014, Zhang et al., 2010), beta (Zhuang et al., 2008), gamma (Bermudez-Hunnaran et al., 2008; Rupa et al., 2008) and IL-10 (Steidler et al., 2000) have been successfully expressed in LAB maintaining their biological active form, producing local and systemic effects after being administrated to mammals.

Although use of Lactococcus lactis strains for delivery of recombinant proteins, such as antigenic proteins, cytokines, including IFN, or other biological active proteins in animals has been previously reported by Zhuang Z, Wu ZG, Chen M, Wang PG. (2008), Bahey-El-Din M, Gahan CG. (2011), W02011150127, W02010139195, CN102329766, CN102796755, CL201503797, CN104120142, CN103074291, US4808523, CN105331570 or W02014040987, the present invention describes a specific solution, of a specific LAB bacteria, to prepare immunomodulating feed to target an unresolved problem in aquaculture, such as bacterial diseases, specifically salmonid diseases in salmon farming.

The use of LAB for in situ delivery of bioactive proteins in Atlantic salmon and rainbow trout has been poorly studied, only exploring its use for delivery of antigenic peptides (Li et al., 2012). The inventors have previously described transformation of Lactococcus lactis bacteria with type I interferon, which proved to be useful preventing the viral disease Infectious Pancreatic Necrosis. However, the present invention describes a strain of Lactococcus lactis transformed with type II IFN that surprisingly could protect fish against bacterial diseases important to aquaculture, specifically salmons. In this invention Lactococcus lactis was used for expressing IFNg from Salmo salar. Lactic bacteria expressing Salmo salar IFNg used in this invention, when administrated with feed, were biologically functional in in vivo testing, protecting against bacterial diseases. Moreover, administration of this modified LAB shows that treated fish can effectively control bacterial infections of intracellular and extracellular pathogens such as Flavobacterium psycrophilum or Pisciricketsia salmonis, which could not have been foreseen or deduced directly from the state of the art.

### SUMMARY OF THE INVENTION

The present invention relates to transformedLactococcus lactis bacteria producing interferon gamma (IFNg) from Salmo salar that comprises the Lactococcus lactis NZ3900 strain that comprises the genetic expression system than comprises the DNA construction pNZ8149-p1-USP45-IFNg-GGG-6xHIS. These transformed Lactococcus lactis bacteria has been assigned the accession number RGM 2416, dated Oct. 22 2017, into INIA's Chilean Collection of Microbial Genetic Resources. Likewise, the invention relates to a plasmid to transform L. lacticbacteria to produce interferon gamma (IFNγ) that comprises the vector pNZ8149 and the DNA sequence P1-USP45-IFNy-GGG-6xHIS and to the method to prepare the previously stated transformed L. lactis bacteria. It is also part of the invention the probiotic feed to immunostimulate fish which comprises transformedL. lactis bacteria with the objective to prevent or treat bacterial infections in fish and the preparation of the feed that comprises mixing the transformed bacteria with fish feed. Furthermore, the invention comprises a fish immunomodulatory composition and a kit that comprises the transformed L. lactis bacteria. Moreover, the invention describes the use of the transformed L. lactis bacteria to prepare feed to immunostimulate fish, to reduce bacterial load, preferably of pathogenic bacteria of fish such as Flavobacterium psychrophilum and/or Piscirickettsia salmonis.

### DESCRIPTION OF THE FIGURES

FIG. 1. Genetic map of the pNZ8149-IFNg construction. PnisA: nisin-inducible promoter. P1: L. lactis constitutive expression promoter. Usp45: secretion signal. IFNy: Salmo salar interferon gamma (IFNγ) mature mRNA coding sequence. 3xGly: sequence coding for three glycines. 6xHis: histidine tail. RepA and RepC: replication genes. LacF: lactase coding gene. Ncol and XbaI restriction sites are shown, these were used to clone the cassette into vector pNZ8149. Plasmid size: 3289 bp.

FIG. 2. Detection of IFN gamma from the cytosolic content of recombinant L. lactis induced with different concentrations of nisin. St: molecular weight standard. Lac: 20 ug of total protein of recombinant L. lactis that contains the pNZ8149 plasmid without the IFNγ gene induced with 10 ng/mL of nisin. 0, 1, 5 y 10: 20 ug of total protein of recombinant L. lactis that contains the pNZ8149 plasmid with the IFNy gene induced with 0, 1, 5 and 10 ng/mL of nisin respectively. To the left, a protein gel dyed with Coomassie blue and to the right a Western Blot for histidine tail (6xHis) detection. The primary antibody used in the Western Blot targeted histidine tails (1:2000), antibodies were incubated for 1 hour at room temperature while shaking. The secondary antibody conjugated to HRP binds to the primary antibody (1:2000) and was incubated for 1 hour at room temperature while shaking. After incubation with each antibody the membrane was washed 3 times for 10 minutes with PBS-Tween10 0.15%. The membrane was developed using the UltraSlgnal® kit and the chemidock LI-COR equipment.

FIG. 3. Secretion kinetics of IFNg produced by L. lactis-IFNy. Four cultures of 40 mL of M17 broth supplemented with lactose were inoculated with 2% of L. lactis-IFNy and incubated at 30° C. without shaking. Supernatant were analyzed at 1, 2 6 and 24 hours after nisin induction. The primary antibody used in the Western Blot targeted histidine tails (1:2000), antibodies were incubated for 1 hour at room temperature while shaking. The secondary antibody conjugated to HRP binds to the primary antibody (1:2000) and was incubated for 1 hour at room temperature while shaking. After incubation with each antibody the membrane was washed 3 times for 10 minutes with PBS-Tweenl0 0.15%. The membrane was developed using the UltraSlgnal® kit and the chemidock LI-COR equipment. C+: 10 ug of cytoplasmic content. To the left the molecular weight standard, in kDa, can be seen. All samples were loaded in duplicate.

FIG. 4. L. lactis-IFNγ IFNγ mRNA copy number quantification using RT-qPCR. Absolute quantification of the number of copies of IFNγ coding mRNA from L. lactis-IFNγ cultures induced with different amounts of nisin was performed. Total RNA was extracted using TRIzol from 20 mL of bacterial culture. Afterwards, 100 ng of RNA from each culture were treated with DNase for RT-qPCR. Reverse transcription controls show no amplification (data not shown). The assay was performed with biological and technical duplicates.

FIG. 5a shows induction of STAT1 of genes related to immune response of SHK-1 cells activated by a sample of sonicated lysate of L. lactis-IFNγ. Different concentrations of samples from mixtures of L. lactis lysates, as presented in Table 1, were used. Lysates were added to SHK-1 cultures for 8 hours at 16° C. Afterwards, induction of STAT1, gamma IP-10, IFNγ, TGF-b, IL-1b and IL-6 was quantified. Obtained values were normalized against eF1a expression. Control: SHK-1 cells without treatment. 0: SHK-1 cell cultures supplemented with 200 ng/mL of L. lactis without insert (IFNγ) extract. 20, 50, 100 y 200: SHK-1 cell cultures supplemented with 0 ng/mL, 20 ng/mL, 50 ng/mL, 100 ng/mL y 200 ng/mL of sonicated extract of L. lactis-IFNγ respectively. The assay was performed with biological and technical duplicates.

FIG. 5b shows induction of gamma IP-10 of genes related to immune response of SHK-1 cells activated by a sample of sonicated lysate of L. lactis-IFNγ. Different concentrations of samples from mixtures of L. lactis lysates, as presented in Table 1, were used. Lysates were added to SHK-1 cultures for 8 hours at 16° C. Afterwards, induction of STAT1, gamma IP-10, IFNγ, TGF-b, IL-1b and IL-6 was quantified. Obtained values were normalized against eF1a expression. Control: SHK-1 cells without treatment. 0: SHK-1 cell cultures supplemented with 200 ng/mL of L. lactis without insert (IFNγ) extract. 20, 50, 100 y 200: SHK-1 cell cultures supplemented with 0 ng/mL, 20 ng/mL, 50 ng/mL, 100 ng/mL y 200 ng/mL of sonicated extract of L. lactis-IFNγ respectively. The assay was performed with biological and technical duplicates.

FIG. 5c shows induction of IFNγ of genes related to immune response of SHK-1 cells activated by a sample of sonicated lysate of L. lactis-IFNγ. Different concentrations of samples from mixtures of L. lactis lysates, as presented in Table 1, were used. Lysates were added to SHK-1 cultures for 8 hours at 16° C. Afterwards, induction of STAT1, gamma IP-10, IFNγ, TGF-b, IL-1b and IL-6 was quantified. Obtained values were normalized against eF1a expression. Control: SHK-1 cells without treatment. 0: SHK-1 cell cultures supplemented with 200 ng/mL of L. lactis without insert (IFNγ) extract. 20, 50, 100 y 200: SHK-1 cell cultures supplemented with 0 ng/mL, 20 ng/mL, 50 ng/mL, 100 ng/mL y 200 ng/mL of sonicated extract of L. lactis-IFNγ respectively. The assay was performed with biological and technical duplicates.

FIG. 5d shows induction of TGF-b of genes related to immune response of SHK-1 cells activated by a sample of sonicated lysate of L. lactis-IFNγ. Different concentrations of samples from mixtures of L. lactis lysates, as presented in Table 1, were used. Lysates were added to SHK-1 cultures for 8 hours at 16° C. Afterwards, induction of STAT1, gamma IP-10, IFNγ, TGF-b, IL-1b and IL-6 was quantified. Obtained values were normalized against eF1a expression. Control: SHK-1 cells without treatment. 0: SHK-1 cell cultures supplemented with 200 ng/mL of L. lactis without insert (IFNγ) extract. 20, 50, 100 y 200: SHK-1 cell cultures supplemented with 0 ng/mL, 20 ng/mL, 50 ng/mL, 100 ng/mL y 200 ng/mL of sonicated extract of L. lactis-IFNγ respectively. The assay was performed with biological and technical duplicates.

FIG. 5e shows induction of IL-1b of genes related to immune response of SHK-1 cells activated by a sample of sonicated lysate of L. lactis-IFNγ. Different concentrations of samples from mixtures of L. lactis lysates, as presented in Table 1, were used. Lysates were added to SHK-1 cultures for 8 hours at 16° C. Afterwards, induction of STAT1, gamma IP-10, IFNγ, TGF-b, IL-1b and IL-6 was quantified. Obtained values were normalized against eF1a expression. Control: SHK-1 cells without treatment. 0: SHK-1 cell cultures supplemented with 200 ng/mL of L. lactis without insert (IFNγ) extract. 20, 50, 100 y 200: SHK-1 cell cultures supplemented with 0 ng/mL, 20 ng/mL, 50 ng/mL, 100 ng/mL y 200 ng/mL of sonicated extract of L. lactis-IFNγ respectively. The assay was performed with biological and technical duplicates.

FIG. 5f shows induction of IL-6 of genes related to immune response of SHK-1 cells activated by a sample of sonicated lysate of L. lactis-IFNγ. Different concentrations of samples from mixtures of L. lactis lysates, as presented in Table 1, were used. Lysates were added to SHK-1 cultures for 8 hours at 16° C. Afterwards, induction of STAT1, gamma IP-10, IFNγ, TGF-b, IL-1b and IL-6 was quantified. Obtained values were normalized against eF1a expression. Control: SHK-1 cells without treatment. 0: SHK-1 cell cultures supplemented with 200 ng/mL of L. lactis without insert (IFNγ) extract. 20, 50, 100 y 200: SHK-1 cell cultures supplemented with 0 ng/mL, 20 ng/mL, 50 ng/mL, 100 ng/mL y 200 ng/mL of sonicated extract of L. lactis-IFNγ respectively. The assay was performed with biological and technical duplicates.

FIG. 6. Feeding outline with IFNγ producing L. lactis in in vivo experiment with rainbow trout. Fish were first acclimated for 7 days prior to treatment; then, a 7 day feeding period began with a) commercial feed, b) L. lactis transformed with an empty plasmid, c) L. lactis-IFNγ. At day 0 (before treatment), 2, 4 and 6 3 fish were sampled. Afterwards, treatment was suspended and all fish were given commercial feed. Finally, 3 fish were sampled from each group at days 1, 3, 5 and 7 days after probiotic feed. (d.p.f) during probiotic feeding and (a.p.f) after probiotic feeding.

FIG. 7a shows RT-qPCR quantification of mRNA related to immune response of interferon gamma (IFNγ) during feeding of Lactococcus lactis-IFNγ to rainbow trout. Relative quantification was performed to different genes that responded to IFNγ. Spleen (SP) and kidney (KD) simples were studied at day 0, 2, 4 and 6 during probiotic feeding (d.p.f) Relative expression of IL-1b was performed normalizing against eF1a. One-tailed Mann-Whitney analysis was performed, and significance was determined by t-student (p<0.05) comparing expression levels to those observed on day 0 before probiotic feeding. NER: Normalized relative expression. Finally, 3 fish were sampled from each group at all timepoints and all assays were performed in technical duplicates.

FIG. 7b shows RT-qPCR quantification of mRNA related to immune response of interferon gamma (IFNγ) during feeding of Lactococcus lactis-IFNγ to rainbow trout. Relative quantification was performed to different genes that responded to IFNγ. Spleen (SP) and kidney (KD) simples were studied at day 0, 2, 4 and 6 during probiotic feeding (d.p.f) Relative expression of IL-12 was performed normalizing against eF1a. One-tailed Mann-Whitney analysis was performed, and significance was determined by t-student (p<0.05) comparing expression levels to those observed on day 0 before probiotic feeding. NER: Normalized relative expression. Finally, 3 fish were sampled from each group at all timepoints and all assays were performed in technical duplicates.

FIG. 7c shows RT-qPCR quantification of mRNA related to immune response of interferon gamma (IFNγ) during feeding of Lactococcus lactis-IFNγ to rainbow trout. Relative quantification was performed to different genes that responded to IFNγ. Spleen (SP) and kidney (KD) simples were studied at day 0, 2, 4 and 6 during probiotic feeding (d.p.f) Relative expression of IL-6 was performed normalizing against eF1a. One-tailed Mann-Whitney analysis was performed, and significance was determined by t-student (p<0.05) comparing expression levels to those observed on day 0 before probiotic feeding. NER: Normalized relative expression. Finally, 3 fish were sampled from each group at all timepoints and all assays were performed in technical duplicates.

FIG. 7d shows RT-qPCR quantification of mRNA related to immune response of interferon gamma (IFNγ) during feeding of Lactococcus lactis-IFNγ to rainbow trout. Relative quantification was performed to different genes that responded to IFNγ. Spleen (SP) and kidney (KD) simples were studied at day 0, 2, 4 and 6 during probiotic feeding (d.p.f) Relative expression of IFNγ was performed normalizing against eF1a. One-tailed Mann-Whitney analysis was performed, and significance was determined by t-student (p<0.05) comparing expression levels to those observed on day 0 before probiotic feeding. NER: Normalized relative expression. Finally, 3 fish were sampled from each group at all timepoints and all assays were performed in technical duplicates.

FIG. 7e shows RT-qPCR quantification of mRNA related to immune response of interferon gamma (IFNγ) during feeding of Lactococcus lactis-IFNγ to rainbow trout. Relative quantification was performed to different genes that responded to IFNγ. Spleen (SP) and kidney (KD) simples were studied at day 0, 2, 4 and 6 during probiotic feeding (d.p.f) Relative expression of STAT1 was performed normalizing against eF1a. One-tailed Mann-Whitney analysis was performed, and significance was determined by t-student (p<0.05) comparing expression levels to those observed on day 0 before probiotic feeding. NER: Normalized relative expression. Finally, 3 fish were sampled from each group at all timepoints and all assays were performed in technical duplicates.

FIG. 7f shows RT-qPCR quantification of mRNA related to immune response of interferon gamma (IFNγ) during feeding of Lactococcus lactis-IFNγ to rainbow trout. Relative quantification was performed to different genes that responded to IFNγ. Spleen (SP) and kidney (KD) simples were studied at day 0, 2, 4 and 6 during probiotic feeding (d.p.f) Relative expression of gIP10 was performed normalizing against eF1a. One-tailed Mann-Whitney analysis was performed, and significance was determined by t-student (p<0.05) comparing expression levels to those observed on day 0 before probiotic feeding. NER: Normalized relative expression. Finally, 3 fish were sampled from each group at all timepoints and all assays were performed in technical duplicates.

FIG. 8. Lysozyme quantification in rainbow trout serum during treatment with L. lactis-IFNγ. Trouts were fed during 7 days with feed supplemented with L. lactis-IFNγ or commercial feed (control). Blood samples were taken at the beginning of the experiment (day 0 d.p.f) and days 2, 4 and 6 during probiotic feed. Serum was obtained from blood samples by centrifuging at 4,500 RPM for 15 minutes at 4° C. The amount of lysozyme was quantified using the Micrococcus lutheus assay observing reduction of optic density, absorbance was measured in a Tecan Pro200 Nanoquant. Finally, 3 fish were sampled from each group at each timepoint. (d.p.f) during probiotic feeding.

FIG. 9a shows RT-qPCR quantification of mRNA related to immune response of interferon gamma (IFNγ) after feeding of Lactococcus lactis-IFNγ to rainbow trout. Relative quantification was performed to different genes that responded to IFNγ. Spleen (SP) and kidney (KD) simples were studied at day 0, 1, 3 and 7 after probiotic feeding (a.p.f). Relative expression of IFNγ (A) was performed using eF1a as normalizing gene and compared results to expression levels on day 0 d.p.f, before probiotic feeding. One-tailed Mann-Whitney analysis was performed, and significance was determined by t-student (p<0.05) comparing expression levels to those observed on day 0 before probiotic feeding. NER: Normalized relative expression. Finally, 3 fish were sampled from each group and all assays were performed in technical duplicates.

FIG. 9b shows RT-qPCR quantification of mRNA related to immune response of interferon gamma (IFNγ) after feeding of Lactococcus lactis-IFNγ to rainbow trout. Relative quantification was performed to different genes that responded to IFNγ. Spleen (SP) and kidney (KD) simples were studied at day 0, 1, 3 and 7 after probiotic feeding (a.p.f). Relative expression of STAT1(B) was performed using eF1a as normalizing gene and compared results to expression levels on day 0 d.p.f, before probiotic feeding. One-tailed Mann-Whitney analysis was performed, and significance was determined by t-student (p<0.05) comparing expression levels to those observed on day 0 before probiotic feeding. NER: Normalized relative expression. Finally, 3 fish were sampled from each group and all assays were performed in technical duplicates. FIG. 9c shows RT-qPCR quantification of mRNA related to immune response of interferon gamma (IFNγ) after feeding of Lactococcus lactis-IFNγ to rainbow trout. Relative quantification was performed to different genes that responded to IFNγ. Spleen (SP) and kidney (KD) simples were studied at day 0, 1, 3 and 7 after probiotic feeding (a.p.f). Relative expression of gIP10 (C) was performed using eF1a as normalizing gene and compared results to expression levels on day 0 d.p.f, before probiotic feeding. One-tailed Mann-Whitney analysis was performed, and significance was determined by t-student (p<0.05) comparing expression levels to those observed on day 0 before probiotic feeding. NER: Normalized relative expression. Finally, 3 fish were sampled from each group and all assays were performed in technical duplicates.

FIG. 9d shows RT-qPCR quantification of mRNA related to immune response of interferon gamma (IFNγ) after feeding of Lactococcus lactis-IFNγ to rainbow trout. Relative quantification was performed to different genes that responded to IFNγ. Spleen (SP) and kidney (KD) simples were studied at day 0, 1, 3 and 7 after probiotic feeding (a.p.f). Relative expression of TGF-β (D) was performed using eF1a as normalizing gene and compared results to expression levels on day 0 d.p.f, before probiotic feeding. One-tailed Mann-Whitney analysis was performed, and significance was determined by t-student (p<0.05) comparing expression levels to those observed on day 0 before probiotic feeding. NER: Normalized relative expression. Finally, 3 fish were sampled from each group and all assays were performed in technical duplicates.

FIG. 9e shows RT-qPCR quantification of mRNA related to immune response of interferon gamma (IFNγ) after feeding of Lactococcus lactis-IFNγ to rainbow trout. Relative quantification was performed to different genes that responded to IFNγ. Spleen (SP) and kidney (KD) simples were studied at day 0, 1, 3 and 7 after probiotic feeding (a.p.f). Relative expression of IL-6 (E) was performed using eF1a as normalizing gene and compared results to expression levels on day 0 d.p.f, before probiotic feeding. One-tailed Mann-Whitney analysis was performed, and significance was determined by t-student (p<0.05) comparing expression levels to those observed on day 0 before probiotic feeding. NER: Normalized relative expression. Finally, 3 fish were sampled from each group and all assays were performed in technical duplicates.

FIG. 10. Lysozyme quantification in rainbow trout serum after treatment with L. lactis-IFNγ. Trouts were fed during 7 days with feed supplemented with probiotic or commercial feed (control) and then fed with commercial feed. Blood samples were taken at days 1, 3, 5 and 7 days after probiotic feeding. Serum was obtained from blood samples by centrifuging at 4,500 RPM for 15 minutes at 4° C. The amount of lysozyme was quantified using the Micrococcus lutheus assay observing reduction of optic density, absorbance was measured in a Tecan Pro200 Nanoquant. Three groups of fish were sampled, the first group was given commercial feed throughout the experiment (control), the second group fish was fed with L. lactis NZ3900 (empty plasmid) and the third group fish was given IFNγ expressing L. lactis (L. lactis IFNγ). Finally, 3 fish were sampled from each group at each timepoint and all assays were performed in technical duplicates. (d.p.f) during probiotic feeding

FIG. 11a shows mortality caused by F. psychrophilum in infected rainbow trout fed with L. lactis IFNγ. Six fish tanks (AQ1-AQ6) were used, each with 15 fish of approximately 25 g. Fish were challenged with F. psychrophilum by intraperitoneal injection of 100 uL (1.25×10⁹ UFC) of inoculum and mortality was observed for 17 days (12° C.). (A) Survival percentage of each studied group. No-challenge Control: fish injected with TYES medium. Flavo: fish infected with F. psychrophilum. Flavo+L. lactis: fish fed for 7 days with L. lactis and then challenged with F. psychrophilum. Flavo+L. lactis IFNγ: fish fed for 7 days with L. lactis expressing IFNγ and then challenged with F. psychrophilum. 100% corresponds to 15 fish per tank.

FIG. 11b shows mortality caused by F. psychrophilum in infected rainbow trout fed with L. lactis IFNγ. Six fish tanks (AQ1-AQ6) were used, each with 15 fish of approximately 25 g. Fish were challenged with F. psychrophilum by intraperitoneal injection of 100 uL (1.25×10⁹ UFC) of inoculum and mortality was observed for 17 days (12° C.). (B) Survival percentage in each fish tank. No-challenge Control: fish injected with TYES medium. Flavo: fish infected with F. psychrophilum. Flavo+L. lactis: fish fed for 7 days with L. lactisand then challenged with F. psychrophilum. Flavo+L. lactis IFNγ: fish fed for 7 days with L. lactis expressing IFNγ and then challenged with F. psychrophilum. 100% corresponds to 15 fish per tank.

FIG. 12. Number of copies of F. psychrophilum RpoS/mL from infected rainbow trout spleen. Trouts were fed with feed supplemented with either commercial feed, L. lactis-IFNγ(IFNγ) or L. lactis (Lactis). Fish were challenged with F. psychrophilum (Flavo) by intraperitoneal injection. Studied groups were FLAVO, IFNγ+FLAVO and Lactis+FLAVO respectively, as negative control (control) fish were fed commercial feed and injected with physiological serum. Using qPCR RpoC was quantified to determine bacterial load in dying and surviving fish after the challenge. Dying fish appeared 7 days after infection and surviving fished were those that did not show signs of dying at day 17 after infection. Bacterial load values below the red line are considered noise associated to the technique.

FIG. 13. Mortality caused by F. psychrophilum in infected rainbow trout fed with L. lactis IFNγ. Twelve fish tanks (AQ1-AQ12) were used, each with 12 fish of approximately 30 g. Fish were challenged with F. psychrophilum by intraperitoneal injection of 100 uL (1.25×10⁹ UFC) of inoculum and mortality was observed for 17 days (12° C.). Control: fish injected with TYES medium. Flavo: fish infected with F. psychrophilum. Flavo +L. lactis: fish fed for 7 days with 1×10⁷ UFC of L. lactis per fish and then challenged with F. psychrophilum. Flavo +L. lactis IFNγ (7da): fish fed for 7 days with 1×10⁷ UFC of L. lactis expressing IFNg and then challenged with F. psychrophilum. Flavo +L. lactis IFNγ (x3): fish fed for 7 days with 3×10⁷ UFC of L. lactis expressing IFNγ and then challenged with F. psychrophilum. Flavo +L. lactisIFNγ (14da): fish fed for 14 days with 1×10⁷ UFC of L. lactis expressing IFNγ and then challenged with F. psychrophilum.

FIG. 14a shwos weight of surviving rainbow trouts in each experimental condition at the end of experiment shown in FIG. 13. Control: fish injected with TYES medium. Flavo: fish infected with F. psychrophilum. Flavo+L. lactis: fish fed for 7 days with 1×10⁷ UFC of L. lactis per fish and then challenged with F. psychrophilum. Flavo +L. lactis IFNγ(7da): fish fed for 7 days with 1×10⁷ UFC of L. lactis expressing IFNγ and then challenged with F. psychrophilum. Flavo+L. lactis IFNg (x3): fish fed for 7 days with 3×10⁷ UFC of L. lactis expressing IFNγ and then challenged with F. psychrophilum. Flavo +L. lactis IFNγ(14da): fish fed for 14 days with 1×10⁷ UFC of L. lactis expressing IFNγ and then challenged with F. psychrophilum. Duplicates are shown as independant experiments.

FIG. 14b shwos length of surviving rainbow trouts in each experimental condition at the end of experiment shown in FIG. 13. Control: fish injected with TYES medium. Flavo: fish infected with F. psychrophilum. Flavo+L. lactis: fish fed for 7 days with 1×10⁷ UFC of L. lactis per fish and then challenged with F. psychrophilum. Flavo+L. lactis IFNγ (7da): fish fed for 7 days with 1×10⁷ UFC of L. lactis expressing IFNγ and then challenged with F. psychrophilum. Flavo+L. lactis IFNg (x3): fish fed for 7 days with 3×10⁷ UFC of L. lactis expressing IFNγ and then challenged with F. psychrophilum. Flavo+L. lactis IFNγ (14da): fish fed for 14 days with 1×10⁷ UFC of L. lactis expressing IFNγ and then challenged with F. psychrophilum. Duplicates are shown as independant experiments.

FIG. 15a shows mortality caused by Piscirickettsia salmonis in infected Salmo salar fed with L. lactis IFNγ. Five experimental conditions, each with a biological duplicate, were studied in 10 fish tanks, each with 8 fish of approximately 45 g. Fish were challenged with P. salmonis by intraperitoneal injection of 100 uL (1×10⁷ live bacteria/mL) of inoculum. (A) Survival percentage in each fish tank. Injection control: fish injected with L15 medium. Challenge control: fish infected by intraperitoneal injection of 100 uL (1×10⁷ live bacteria/mL) P. salmonis. L. lactis+SRS: fish infected by intraperitoneal injection of 100 uL (1×10⁷ live bacteria/mL) P. salmonis, fed for 7 days L. lactis and given a 3-day booster. L. lactis IFNγ (IFNg) (7da) +SRS: fish infected by intraperitoneal injection of 100 uL (1×10⁷ live bacteria/mL) P. salmonis, fed for 7 days L. lactis IFNγ and given a 3-day booster. L. lactis IFNγ (IFNγ) (14da) +SRS: fish infected by intraperitoneal injection of 100 uL (1×10⁷ live bacteria/mL) P. salmonis and fed for 14 days L. lactis IFNγ. Results are shown in duplicates.

FIG. 15b shows mortality caused by Piscirickettsia salmonis in infected Salmo salar fed with L. lactis IFNγ. Five experimental conditions, each with a biological duplicate, were studied in 10 fish tanks, each with 8 fish of approximately 45 g. Fish were challenged with P. salmonis by intraperitoneal injection of 100 uL (1×10⁷ live bacteria/mL) of inoculum. (B) Survival percentage per studied condition. Injection control: fish injected with L15 medium. Challenge control: fish infected by intraperitoneal injection of 100 uL (1×10⁷ live bacteria/mL) P. salmonis. L. lactis+SRS: fish infected by intraperitoneal injection of 100 uL (1×10⁷ live bacteria/mL) P. salmonis, fed for 7 days L. lactis and given a 3-day booster. L. lactis IFNγ (IFNg) (7da) +SRS: fish infected by intraperitoneal injection of 100 uL (1×10⁷ live bacteria/mL) P. salmonis, fed for 7 days L. lactis IFNγ and given a 3-day booster. L. lactis IFNγ (IFNγ) (14da) +SRS: fish infected by intraperitoneal injection of 100 uL (1×10⁷ live bacteria/mL) P. salmonis and fed for 14 days L. lactis IFNγ. Results are shown in duplicates.

FIG. 16. Mortality caused by Piscirickettsia salmonis in infected Salmo salar fed with L. lactis transformed with an empty plasmid. Three experimental conditions, each with a biological duplicate were studied in 6 fish tanks, each with 7 fish of approximately 50 g. Fish were fed for 7 days and then challenged with P. salmonis by intraperitoneal injection. Vehicle control: fish injected with L15 medium. P. salmonis: fish injected with P. salmonis (1×10⁷ live bacteria/fish) of inoculum. P. salmonis+pNZ8149 w/o insert: Salmo salar fed with feed supplemented with L. lactis transformed with the vector pNZ8149 without S. salar's IFNγ gene and challenged with P. salmonis (1×10⁷ live bacteria/fish). Graphs show average survival percentage in each condition.

### DETAILED DESCRIPTION

The present invention refers to a probiotic feed that comprises lactic acid bacteria, particularly L. lactis, transformed to produce an immunostimulant cytokine, preferably type II IFN, to immunostimulate aquatic species against bacterial infections, thus achieving heterologous expression of an immunostimulant protein of Salmo salar in lactic acid bacteria. The L. lactisbacteria has been assigned the accession number RGM 2416, dated Oct. 22, 2017, by the INIA's Chilean Collection of Microbial Genetic Resources, Chile.

Immunostimulant proteins strongly regulate immune response. Administration of recombinant immunostimulant proteins have allowed their use as a therapeutic to increase immune response against bacterial pathogens. In aquaculture, there are no available in situ heterologous production systems for functional immunostimulant proteins for therapeutic use. Lactic acid bacteria are GRAS (Generally Regarded as Safe) organisms, that because of this classification have been used as vaccines and as therapeutic release systems.

The lactic acid bacteria of this invention can be used to express and secrete functional immunostimulant proteins in aquatic species, particularly in fishes and preferably in Salmo salar and rainbow trout. The present lactic acid bacteria allows non-invasive immunogenic protection applicable in large scale, capable of increasing current immunization systems.

Administration of the present lactic acid bacteria, that produce an immunostimulant cytokine, can stimulate in vivo the expression of genes that respond to IFN gamma and reduce mortality of aquatic species challenged with F. psychropillhum and P. salmonis

Using molecular biology tools, lactic acid bacteria was generated that produce an immunostimulant cytokine. Its immunostimulant properties were studied in species highly important for aquaculture, administrating an oral dosage of 1×10⁷ UFC per fish. Immunostimulation was assessed quantifying genes that respond to this cytokine using real time qPCR normalizing their expression against the housekeeping gene eF1a. To achieve this objective, total RNA was extracted from immunological organs (spleen and kidney), which were previously assessed studying their morphology and any signs of toxicity. The lactic acid bacteria produce and secret the heterologous protein. The protein was located mainly in the cytoplasmatic fraction. The bioassays showed that the protein is functional when stimulating the expression of immunological genes.

The invention relates, particularly, to transformed Lactococcus lactis bacteria that produce interferon gamma from Salmo salar that includes the DNA construction that comprises pNZ8149-P1-USP45-IFNg-GGG-6xHIS where pNZ8149 is the transformed vector; P1 is L. lactis constitutive expression promoter; Usp45 is a secretion signal; IFNγ is Salmo salar interferon gamma (IFNγ) mature mRNA coding sequence; GGG is a sequence coding for three glycines and 6xHis is s sequence coding for 6 terminal histidines. The transformed strain of Lacococcus lactis is identified as NZ3900, however, it is not limiting and other strains of L. lactis could be used. The bacteria is preferably Lactococcus lactis NZ3900 transformed with the genetic system that comprises the plasmid pNZ8149/P1-USP45-IFNg-GGG-6xHIS. Preferably, the recombinant strain is the one with the accession number RGM 2416, dated Oct. 22, 2017, by the INIA's Chilean Collection of Microbial Genetic Resources, Chile.

It is also part of the invention, a useful plasmid to transform L. lactis bacteria to produce interferon gamma (IFNγ) that comprising the vector pNZ8149 and the sequence P1-Usp45-IFNγ-GGG-6xHIS; where P1 is L. lactis constitutive expression promoter; Usp45 is a secretion signal; IFNγ is Salmo salar interferon gamma (IFNγ) mature mRNA coding sequence; GGG is a sequence coding for three glycines and 6xHis is s sequence coding for 6 terminal histidines.

Likewise, the invention relates to a method to prepare the transformed L. lactis bacteria that comprises the following steps:
a) Digesting with restriction enzymes a plasmid that contains the reading frame of Salmo salar IFNg codon optimized for Lactococcus lactis,
b) ing the digestion product, corresponding to the IFNg gene with sticky ends for restriction enzymes,
c) Meanwhile, digesting with the same enzymes the plasmid NZ8149,
d) Purifying the linearized plasmid from an agarose gel,
e) Ligating both purified products with ligase,
f) Dialyzing the ligation product,
g) Transforming electrocompetent L. lactis bacteria with the ligated plasmid.

The restriction enzymes are preferably, for steps a) and c) restriction enzymes Ncol and Xbal and electrocompetent bacteria of step g) is the strain L. lactis NZ3900.

It is part of this invention a probiotic feed to immunostimulate aquatic species, preferably immunostimulate fishes. The probiotic feed comprises transformed L. lactis bacteria with a transformed Lactococcus lactis bacteria producing interferon gamma of Salmo salar that includes the DNA construction that comprises pNZ8149-P1-USP45-IFNg-GGG-6xHIS where pNZ8149 is the transformed vector; P1 is L. lactis constitutive expression promoter; Usp45 is a secretion signal; IFNγ is Salmo salar interferon gamma (IFNγ) mature mRNA coding sequence; GGG is a sequence coding for three glycines and 6xHis is s sequence coding for 6 terminal histidines. Likewise, the invention comprises the method to prepare the probiotic feed described above.

Additionally, this invention protects any immunomodulatory composition for aquatic species, under the broadest scope of composition. The composition can be liquid or solid and have excipients to provide stability for storage and enhance bioavailability of the transformed bacteria of this invention. Furthermore, the composition includes all combinations of useful organic molecules to administrate to aquatic species such as proteins, lipids, saccharides, in combination with the transformed L. lactis bacteria described above or with the bacteria described in the preferred conducted examples. This composition can comprise the combination of the bacteria of the invention with recombinant protein vaccines or with nucleic acid vaccines, bacterines, probiotics, prebiotics or other immunomodulators, vitamins, etc.

The use of the transformed L. lactis bacteria is for preparing feed or a useful composition for immunostimulating aquatic species, particularly, to immunostimulate fishes. The given use for these bacteria is to prepare useful feed to reduce bacterial load, preferably bacterial load of Flavobacterium psychrophilum and or Piscirickettsia salmonis. The use of the bacteria and the feed that comprises it is to prepare a feed or a useful composition to treat or prevent infection from Flavobacterium psychrophilum and/or Piscirickettsia salmonis in fishes.

Is part of this invention, a kit that includes a packing that comprises the transformed L. lactisbacteria with the DNA construction pNZ8149-P1-USP45-IFNg-GGG-6xHIS. Likewise, this kit can comprise instructions to be used to feed, treat or prevent bacterial diseases in aquatic species, preferably, fishes important for aquaculture. In the preferred embodiment instructions have information for the use for feeding fishes, treat or prevent diseases caused by Flavobacterium psychrophilum and Piscirickettsia salmonis in fishes.

The invention also comprises the method to reduce bacterial load in aquatic species, that comprises the administration to this species the feed prepared with transformed L. lactisbacteria, selected from any bacteria that comprises the DNA construction pNZ8149-P1-USP45-IFNg-GGG-6xHIS detailed in the examples of this invention. The aquatic species are preferably fishes, and the method treats or prevents infection with Flavobacterium psychrophilum or Piscirickettsia salmonis. The method for delivery is capable of treating or preventing a bacterial infection by reducing bacterial load in aquatic species, preferably fishes.

### EXAMPLES

Example 1: Using synthetic biology the coding gene for a codon-optimized immunostimulating protein from Salmo salar with a secretion peptide and 6 histidine tail was designed and synthesized. The gene was clones into a food grade expression vector. Production of the heterologous protein was confirmed using Western Blot and its functionality through bioassays.

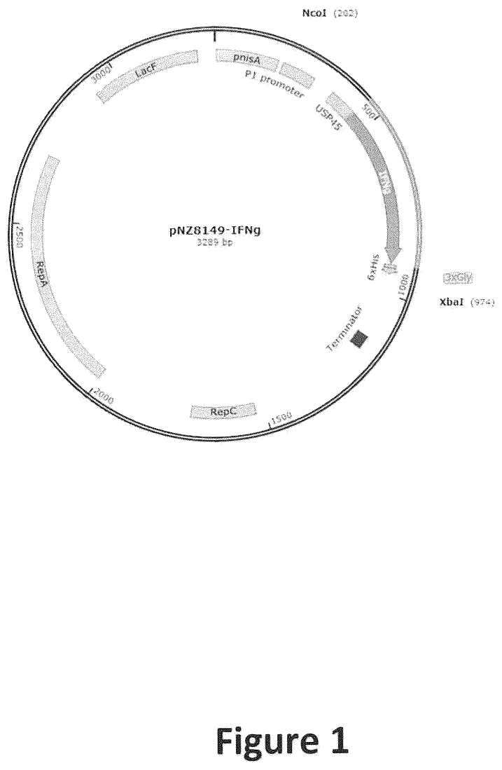

The present invention consists in a recombinant Lactococcus lactis NZ3900 strain, that comprises the plasmid NZ8149 into which a synthetic DNA segment, that comprises P1 promoter, the signal peptide of protein USP45, a gene that codifies the mature sequence of type I interferon of Salmo salar and a tail of 3 glycines and 6 histidines, denominated as pNZ8149-IFNgSS (FIG. 1), was cloned.

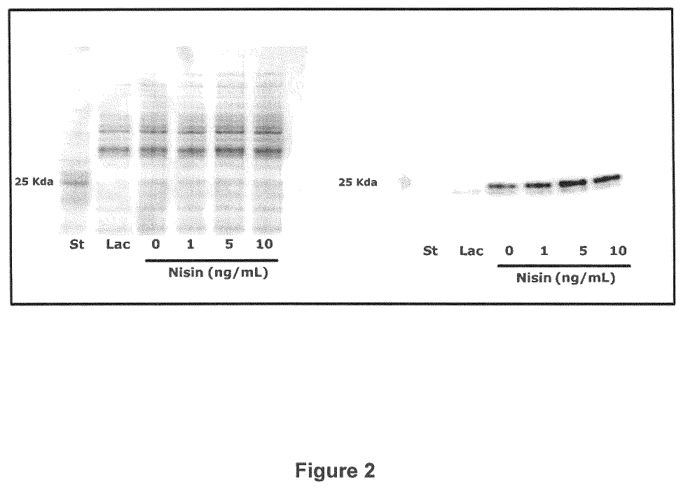

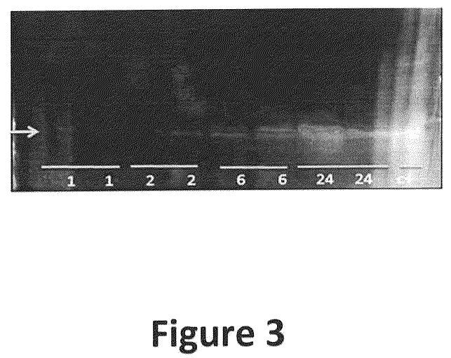

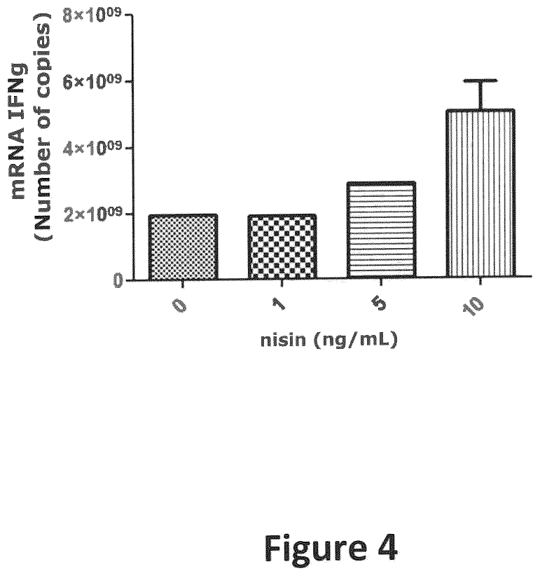

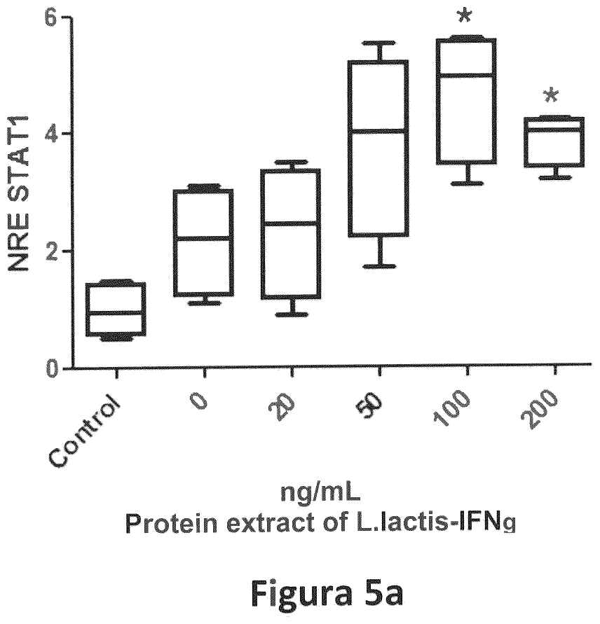

The sequence of the IFNγ gene was obtained from Salmo salar's genome and codon-optimized to be expressed in an L. lactis host (FIG. 1). This strain expresses constitutively the IFNγgene driven by promoter P1, from L. lactis sequence SEQ IDNo:2, and in inducible by the nisin promoter present in the commercial vector (FIG. 2). IFNγ is secreted to the culture media by the signal peptide Usp45, sequence SEQ ID No3, that is merged in the reading frame of the IFN gamma sequence, SEQ ID No:4 (FIG. 3). Expression of IFNγ was detected in extracts of IFNγ producing L. lactis extract compared to extracts of the strain that contains the pNZ8149 without the gene's reading frame (FIG. 4).

### Methodology

Design of the pNZ8149 vector. To design the lactococcal vector, the plasmid pNZ8140 (Mobitec®) was used as backbone and the plasmid pJet1.2-IFNg (Genescript®) contained the reading frame of Salmo salar's IFNγ (GenBank: FJ263446.1). The IFNγ gene was codon-optimized in silico for Lactococcus lactis NZ3900 using the online application www.kazusa.or.jp, and then modified to include in the amino terminal end the sequence that codifies the USP45 peptide and in the carboxylic end a sequence that codifies a tail of 3 glycines and 6 histidines (GGGHHHHHH) SEQ ID No:5. These sequences were added in frame with the L. lactis codon-optimized IFNγ gene. Likewise, the P1 promoter sequence was added in silico to the 5′ end of the sequence that codifies USP45-IFNg-GGGHHHHHH. This new sequence, P1-USP45-IFNg-GGGHHHHHH was synthetized in vitro and cloned into pJet1.2 (Genescript®) (FIG. 1). The identity of the genetic sequence of the construct was corroborated by Sanger sequencing and the amino acid sequence that the gene synthetizes using the web page www.bioinformatics.org.

Because the plasmids pNZ8149 and pJet1.2-IFNγ have a replication origin for propagation in E. coli and ampicillin resistance as selection marker, the MC1061 strain was used to obtain high concentrations of both plasmids. Afterwards, both plasmids were digested for 1 hour at 37° C. with enzymes Ncol and Xbal, present in the multiple cloning site of pNZ8149 and in each end of the IFNγ in pJet1.2. Both digestions were corroborated in a 1% w/v agarose gel from which the linear vector and the insert were purified using the Wizard SV Gel and PCR Clean-Up System (Promega®). The vector and the insert were ligated overnight at 4° C. and dialyzed the next day for 30 minutes prior to electroporation with Lactococcus lactis NZ3900 to obtain the bacteria with vector pNZ8149-IFNg.

Detection of IFNγ in L. lactis cultures. To detect the expression of the recombinant protein, Western Blot assays were performed from cytoplasmatic extract of sonicated L. lactis. To do so, a preculture of L. lactis pNZ8149-IFNg was cultured overnight at 30° C. in M17 media with 0.5% lactose. The following day, a 40 mL culture was inoculated with 2% inoculum, after cultivating at 30° C. up to an OD₆₀₀ 0.4-0.6 the culture was divided in equal parts. To some cultures, nisin was added between 0 and 10 ng/mL. Cultures were then incubated for 2 hours at 30° C. Afterwards, bacteria were collected through centrifugation at 7,000 RPM for 20 min at 4° C.

The bacterial pellet was resuspended in 500 uL of PBS1x supplemented with a protease inhibitor 1 mM. Afterwards, it was sonicated 5 times for 15 seconds each with an Ultrasonic processor Sonic Vibracell VCX130 (90% amplitude), kept in ice between cycles. The sample was centrifuged at 6,000 RPM for 10 min at 4° C. and the supernatant was separated from the cellular debris, storage in a new tube and frozen at −20° C. until its use.

To determine total protein concentration in the extract the Bradford method was used. Once the concentration was known, it was normalized to resolve and compare the amount of protein through SDS-PAGE electrophoresis. 10 ug of the cytoplasmatic extract of induced and not induced with nisin IFNγ producingL. lactis and of L. lactis that contained a pNZ8149 plasmid without a IFNγ reading frame as control were loaded. Samples were run in an electrophoresis for 40 min at 60 V and then for 1.5 hr at 120 V. Proteins contained in the polyacrylamide gel were transferred to a nitrocellulose membrane at 300 mA for 7 min. Afterwards, the membrane was blocked with a PBS-BSA 2% solution while shaking at 4° C. Then, the membrane was washed at room temperature 3 times for 10 minutes with PBS 1X-Tween 20 at 0.1% and incubated for 1 hour with an anti-His primary antibody at a 1:2,000 dilution in a 2% PBS-BSA solution. Next, the membrane was washed with the same procedure and then incubated for 1 hour with a 1:5,000 dilution of the secondary antibody, an anti-rabbit IgG conjugated with horseradish peroxidase. The membrane was washed again and developed by chemiluminescence with the Supersignal® West Pico Chemilumminescent Substrate Kit, incubating the solutions for 5 minutes and exposing the membrane y a digital development equipment C-Digiy model 3600 (LI-COR) for 7 minutes for imaging.

Detection of IFNγ in the supernatant of L. lactis cultures. To detect the protein from the supernatant of L. lactis cultures, the supernatant was collected after centrifuging the bacterial culture, then it was precipitated with 6 volumes of acetone and stored at −20° C. overnight. Afterwards, it was centrifuged at 6,000 RPM for 10 min at 4° C., then the supernatant was discarded and the precipitate was washed 2 times with double distilled water, finally, the pellet was resuspended in 50 uL of protein lading buffer. To detect the protein the Western Blot protocol described above was used.

IFNγ mRNA quantification in L. lactis cultures. To correlate the induction of protein with IFNγ messenger RNA levels generated by L. lactis (FIG. 4), culture and induction conditions were replicated. After nisin induction for 2 hours, the bacterial culture was centrifuged at 6,000 RPM at 4° C. for 10 minutes, the supernatant was discarded and the bacteria was lysed in 1 mL of TRIzol to extract total RNA according to the manufacturer's recommendations. Afterwards, RNA was treated with DNase I (Promega®) to remove traces of contaminant plasmidial DNA, then this was used to quantify the number of IFNγ coding mRNA copies by qRT-PCR. The reaction mix was: 2 uL of total RNA (100 ng), 5 uL od 2X SensiMix SYBR No-ROX One-Step Kit, 0.5 uL of IFNγ L. lactis Fw primer, 0.5 uL of IFNγ L. lactis Rv primer (10 mM) (Table 2) and 2 uL of nuclease free water.

Biological activity of IFNγ produced by L. lactis. To determine if the produced protein was biologically active, cytoplasmatic extracts of the bacteria were incubated with SHK-1 cells, cells derived from Salmo salar (Table 1) and then some transcripts involved in the response to IFNγ were quantified. Briefly, different proportions between L. lactis and L. lactis-IFNγ cytoplasmatic extracts were mixed with L-15 medium and incubated for 8 hours at 16° C. with SHK-1 cells to determine the contribution of the recombinant protein amount from the total protein content by the bacteria. Afterwards, total RNA from salmon cells was extracted using the E.Z.N.A Total RNA kit (OMEGA bio-tek) and treated with DNase I (Promega®). Treated RNA was used for synthesis of cDNA with Oligo(dT) with the following reaction mix: 11 uL of treated RNA, 0.5 uL of Oligo(dt) (10 mM), 1 uL of dNTPs (10 mM each), 0.2 uL of M-MLV (Promega C)), 7 uL of nuclease free water. The obtained cDNA was diluted 1:2 and used for relative quantification of transcripts related to the immune response by qPCR with the following reaction mix: 2 uL of cDNA, 5 uL of 2X SensiMix SYBR No-ROX Kit, 0.25 uL of Fw (10 mM),0.25 uL of Rv (10 mM) and 2 uL of nuclease free water (Table 2).

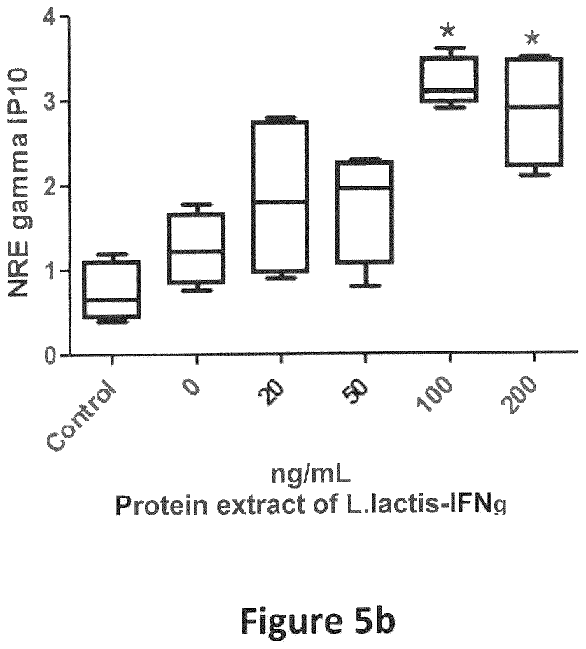

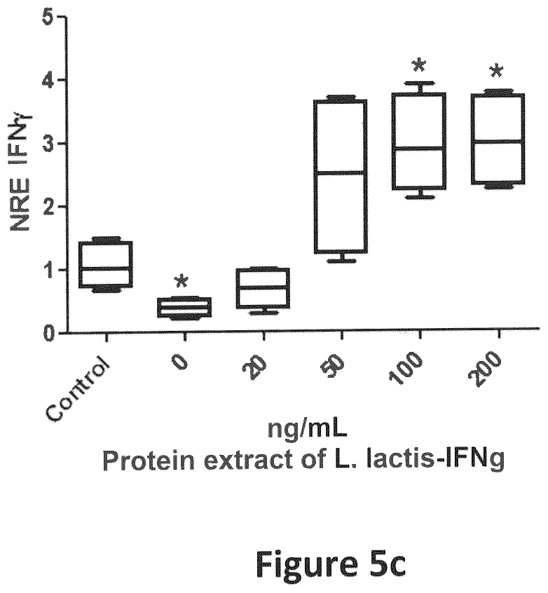

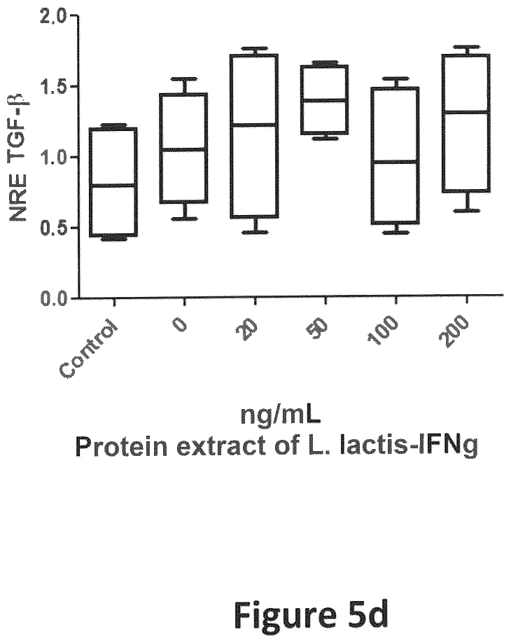

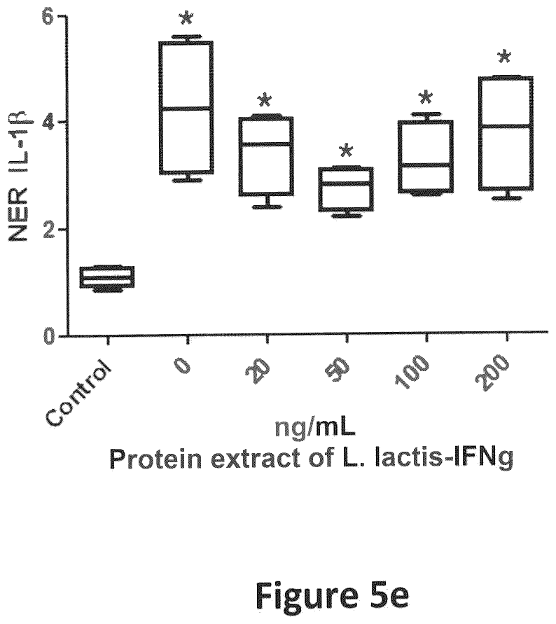

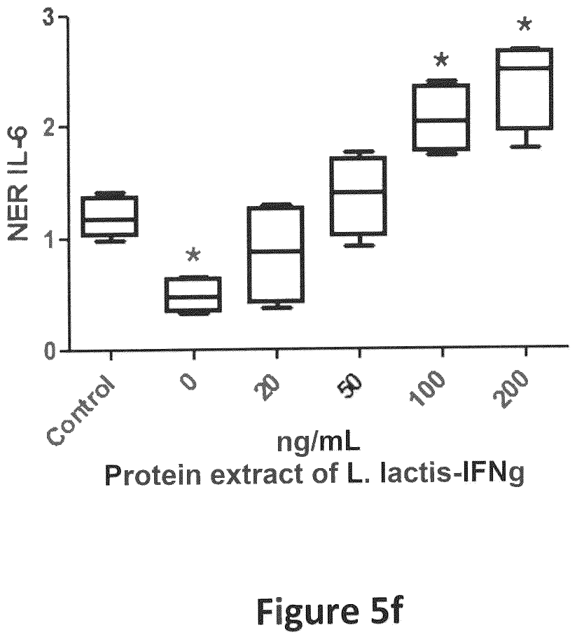

TABLE 1
(FIG. 5) Amount of total protein used in assays to
determine the contribution of IFNγ to the immune
response of SHK-1 cells. Samples with 20 ug of
total protein amount were obtained by mixing
sonicated lysate of IFNγ expressing L. lactis
(L.L IFNγ) and lysate of L. lactis that does not
express it (L.L empty) in different proportions.
Starting from the sample containing 20 ug of
total protein all working dilutions were prepared.

| L.L IFNγ | L.L empty | Final protein |
|---|---|---|
| (ug) | (ug) | content (ug) |
| 0.0 | 20.0 | 20 |
| 2.0 | 18.0 | 20 |
| 6.0 | 14.0 | 20 |
| 10.0 | 10.0 | 20 |
| 14.0 | 6.0 | 20 |
| 20.0 | 0.0 | 20 |

TABLE 2
Nucleotide sequence of primers used for quantification of immune
response genes and studied pathogens.

| Gene | Primer | 5′ → 3′ |
|---|---|---|
| IFNγ salmon | FW | CCG TAC ACC GAT TGA GGA CT |
| | RV | GCG GCA TTA CTC CAT CCT AA |
| IL-1b salmon | FW | CCC CAT TGA GAC TAA AGC CA |
| | RV | GCA ACC TCC TCT AGG TGC AG |
| TGF-β salmon | FW | AGC TCT CGG AAG AAA CGA CA |
| | RV | AGT AGC CAG TGG GTT CAT GG |
| gamma IP10 salmon | FW | GTG TCT GAA TCC AGA GGC TCC A |
| | RV | TCT CAT GGT GCT CTC TGT TCC A |
| IFNγ | FW | CAC ATT TGC AAA ATC TTT GGG CT |
| L. lactis-IFNγ | RV | CAA TCG TTG TGC TTG TCG TCT |
| IL-6 salmon | FW | CCT TGC GGA ACC AAC AGT TTG |
| | RV | CCT CAG CAA CCT TCA TCT GGT C |
| IL-12 salmon | FW | TGA CGC TTT TTC TCA CCG GTT GT |
| | RV | ACG CTT TGC AGC ATG AGC TTG A |
| eF1α salmon | FW | GGG TGA GTT TGA GGC TGG TA |
| | RV | TTC TGG ATC TCC TCA AAC CG |
| RpoS F. | FW | GAA GAT GGA GAA GGT AAT TTA GTT |
| psychrophilum | | GAT ATT |
| | RV | CAA ATA ACA TCT CCT TTT TCT ACA ACT |
| | | TGA |
| STAT1 | FW | GAC CAG CGA ACC CAA GAA CCT GAA |
| | RV | CAC AAA GCC CAG GAT GCA ACC AT |
| rDNA 16S | FW | AGG GAG ACT GCC GGT GAT A |
| P. salmonis | RV | ACT ACG AGG CGC TTT CTC A |

### Result Description

pNZ8149 has a food grade selection marker that allows lactose metabolism (LacF), it has a constitutive promoter P1 and a nisin inducible promoter, L. lactis' natural secretion signal Usp45, Salmo salar IFNγ codifying gene and a histidine tag. RepA and RepC are genes that allow the plasmid's replication (FIG. 1). When transforming NZ3900 bacteria, which presents a chromomsomal deletion of the lactose ORF, the bacteria can use this carbon sourse and keep the plasmid. The constitutive promoter P1 allows the expression of IFNγ, this induction can be proportionaly increased with the amount of nisin added to culture media due to the pNisA promoter (FIG. 2). The protein can be easily detected from the cytoplasmatic fraction by Western Blot, however the amount of secreted protein is in a much lower proportion (FIG. 3). This increase in the amount of protein detected by Wester Blot can be seen when quantifying the number of transcripts of IFNγ mRNA by qRT-PCR (FIG. 4).

When using the cytoplasmitic extract of sonicated bacteria on salmon SHK-1 cell line, normalizing the amount of total protein (Table 1) it is possible to detect the induction of transcipts such as STAT1, gIP10 and IL-1b, suggesting that the protein is functional in vitro because the first two are involved in the signalling cascade initiated by IFNγ and the third is involved in the natural proimmflamatory response following IFNg. (FIG. 5). However, when quantifying IL-6, a pleitropic cytokine related to an induced innate immune response, it increases proportionally with the amount of recombinant protein present in culture, surprisingly suggesting that IFNγ would be inducing its expression.

### Example 2: Effects of administering IFNγ producig L. lactis bacteria

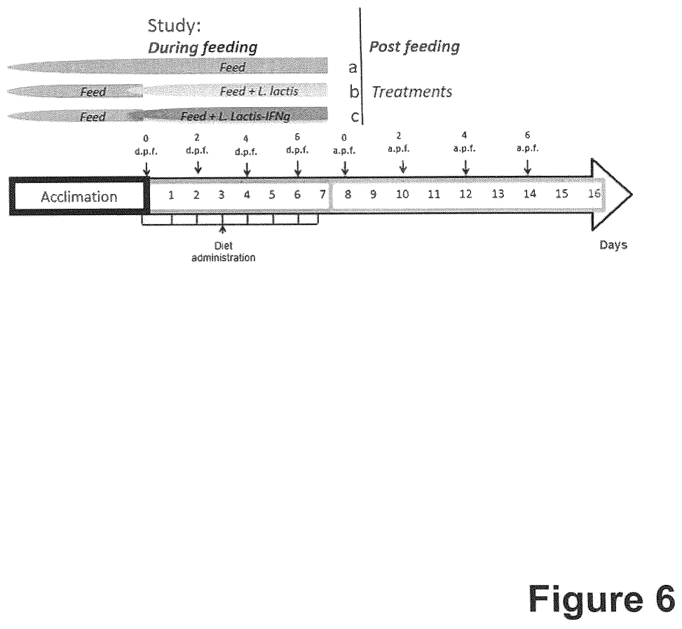

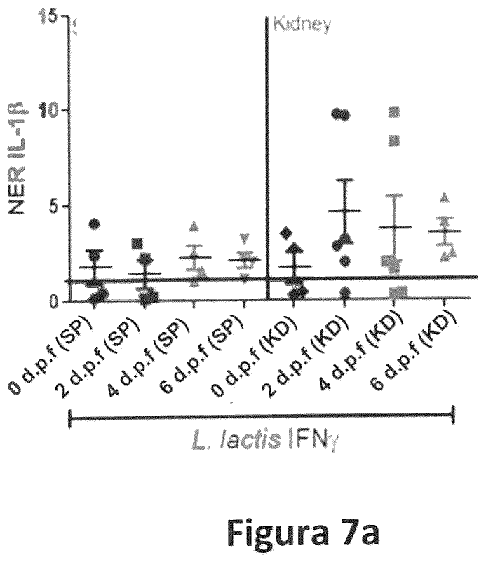

To assess the immunoestimulating effect of IFNγ producing L. lactis in an in vivo model an assay was performed with rainbow trouts (average weight: 20-25 g) were the effect of the probiotic was evaluated through the course of the experiment during and after feeding it as it is described in FIG. 6.

The approach consisted in acclimating the fish for 7 days, then feeding them for 7 days with a) L. lactis-IFNγ, b) L. lactis with an empty vector and c) commercial feed. At day 0 (before treatment), 2 and 6 and 3 fish were sampled. Afterwards, treatment was suspended and all fish were given commercial feed. Then, 3 fish were sampled from each group at days 1, 3, 5 and 7 days after probiotic feed. While sampling, during probiotic feed (d.p.f) and after probiotic feed (a.p.f), 100 uL of blood was extracted from 3 fish, which were then sacrificed prior to spleen and kidney extraction. Blood was used to quantify lysozyme enzymatic activity in serum by means of Micrococcus luteus suspensions. The assay consisted in measuring the decrease of absorbance at 450 nm in 180 uL of a M. luteus stock incubated with 20 uL of serum, using as a positive control 200-400 units of lysozyme/mL.

The following formula was used to calculate the amount of lysozyme:

$$units / ml enzyme = \frac{( Δ {A}_{450} / min Sample - Δ {A}_{450} / min Blanc )}{( 0.001 ) ( 0.02 )}$$

To determine if stimulation of the immune response at a transcript level occurs, total RNA from spleen and kidney was extracted with TRIzol following the manufacturers recommendation. Then, cytokines IL-1β, IL-6, IL-12, TGF-β, IFNγ, gamma IP10 and STAT1 were quantified by relative quantification. Treated RNA with DNase I was used as template for cDNA synthesis with Oligo(dT) following this reaction mixture: 11 uL od treated RNA, 0.5 uL of Oligo(dt) (10 mM), 1 uL of dNTP (10 mM each), 0.5 uL of M-MLV (Promega®), 7 uL of nuclease free water. The obtained cDNA was diluted 1:2 and used as template for relative quantification of the transcripts related to the immune response by qPCR with the following reaction mix: 2 uL of cDNA, 5 uL of 2X SensiMix SYBR No-ROX Kit, 0.25 uL of Fw (10 mM), 0.25 uL of Rv (10 mM) and 2 uL of nuclease free water (Table 2).

To assess if the observed immunostimulation is able to create protection against pathogens relevant to salmon farming, a challenge in rainbow trouts (average weight: 15-20 g) was set up using F. psychrophilum (1×10⁸ bacteria/fish) as model. Fish were acclimated for 7 days prior to treatment; then, a 7 day special feeding period began and 5 days later the challenge was performed. Afterwards, mortality was registered during the following 18 days to asses immune response and bacterial load.

In this assay the following treatments were studied: a) Fish fed for 7 days with L. lactis-IFNγ(1×10⁷ bacteria/fish) and then infected, b) Fish fed for 7 days with L. lactis with an empty vector (1×10⁷ bacteria/fish) and then infected and c) Fish fed for 7 days with commercial feed and then infected. Mortality was registered for 17 days after infection, time during which spleen of dying fish was extracted and from fish that survived the experiment for bacterial load quantification

To determine if higher doses or a prolonged diet could increase survival, a second challenge in rainbow trout (average weight: 30-40 g) was performed with F. psychrophilum (1×10⁸ bacteria/fish). In this assay, fish were acclimated for 7 days, then special feeding began for 7 days and 5 days afterwards the challenge was performed. The following conditions were studied: a) Fish fed for 7 days with a doses 3 times higher than the original of L. lactis-IFNγ(3×10⁷ bacteria/fish) and then infected, b) Fish fed for 13 days with L. lactis-IFNγ (1x10 7 bacteria/fish) and then infected, c) Fish fed for 7 days with L. lactis-IFNγ (1×10⁷ bacteria/fish) and then infected, d) Fish fed for 7 days with L. lactis with and empty plasmid (1×10⁷ bacteria/fish) and then infected and e) Fish fed for 7 days with a commercial feed and then infected. To determine if probiotic administration changes the physical condition, length and weight of surviving fish was recorded.

To assess the protective effect of L. lactis-IFNγ on Salmo salar, the intracellular bacteria Pisiricketssia salmonis was used as a model (1×10⁷ bacteria/fish). Fish were first acclimated for 7 days and then the following treatments began: a) Fish fed for 7 days L. lactis-IFNγ (1×10⁷ bacteria/fish) and then infected, b) Fish fed for 14 days L. lactis-IFNγ (1x10 7 bacteria/fish) and then infected, c) Fish fed for 7 days with L. lactis with and empty plasmid (1x10 7 bacteria/fish) and then infected and d) Fish fed for 7 days with commercial feed and then infected. Mortality was recorded for 23 days after infection.

Bacterial Load: Bacterial load of infected fish with P. salmonis or F. psycrophillum was determined by absolute qPCR using total DNA extracted from the immunological organs spleen and kidney. From these organs, total DNA was extracted with the Wizzart Genomic DNA Kit (Promega C)). Purified DNA was quantified by absorbance at 260 nm. 50 ng of total DNA were used for the qPCR reaction. To detect P. psycrophillum the primers rpoS-F (5′ GAA GAT GGA GAA GGT AAT TTA GTT GAT 3′) and rpoS-R (5′ CAA ATA ACA TCT CCT TTT TCT ACA ACT 3′) were used, which amplify a 200 bp fragment. To detect P. salmonis the primers 16SPS-F (5′ AGG GAG ACT GCC GGT GAT A 3′) and 16SPS-R (5′ ACT ACG AGG CGC TTT CTC A 3′) were used. Each amplification product showed only one denaturation peak indicating a specific amplification product. For each one of the amplicons a calibration curve was constructed with increasing concentrations of the PCR product previously cloned on pGEM-T. Through this method the number of bacteria was calculated, assuming that there is only one copy of the gene in each bacterium chromosome.

### Result Description

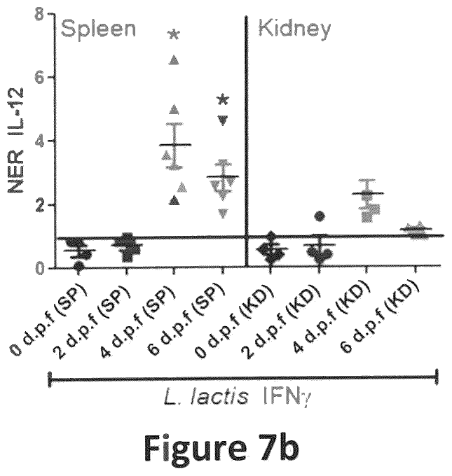

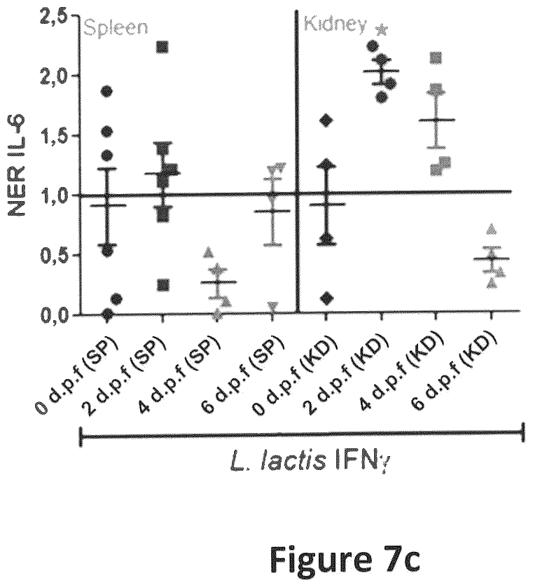

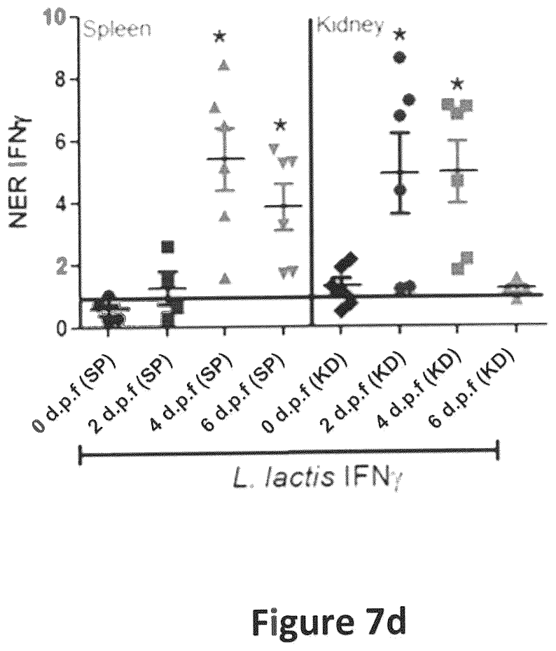

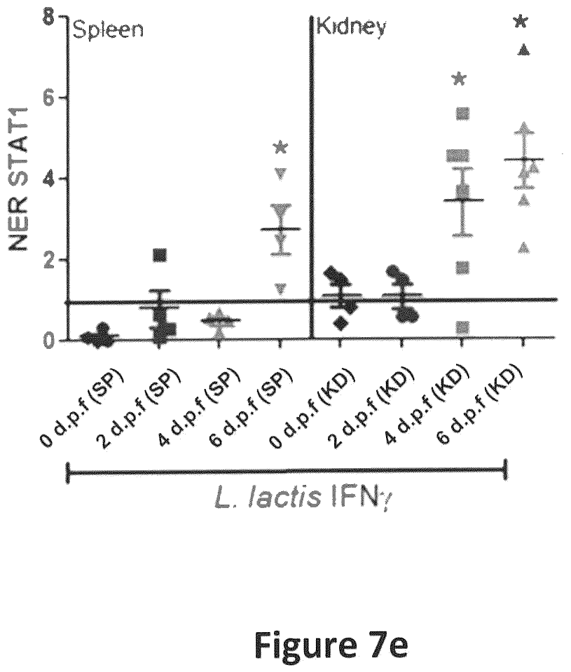

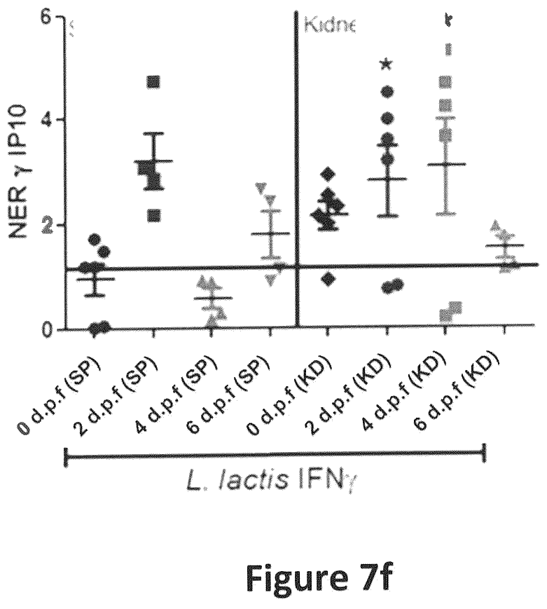

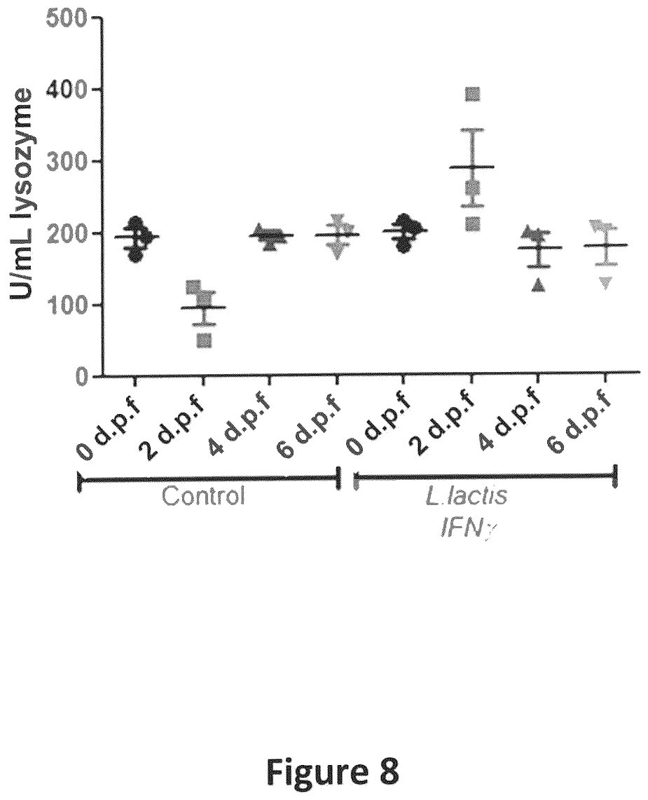

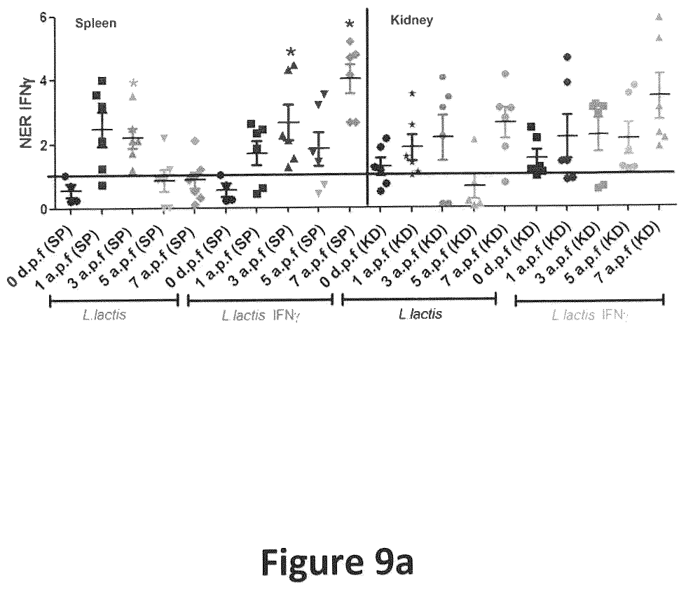

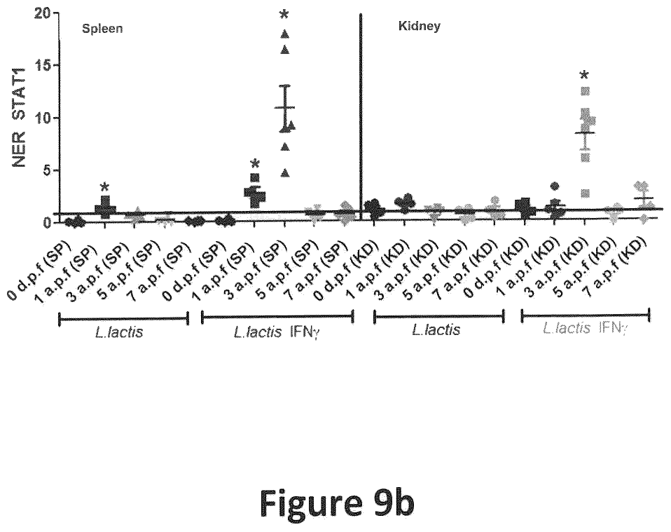

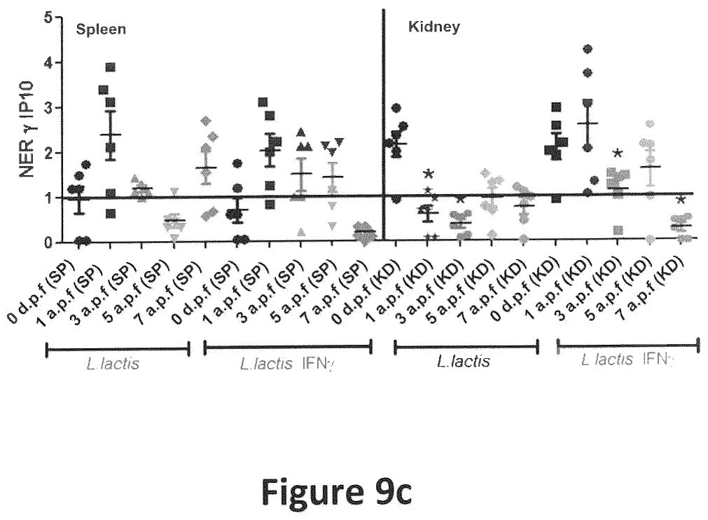

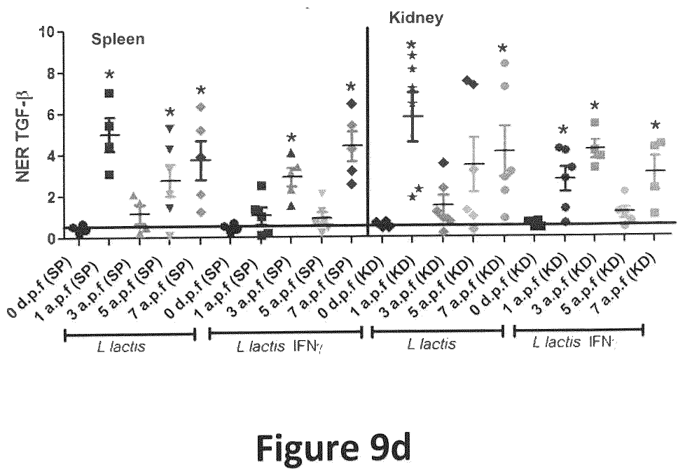

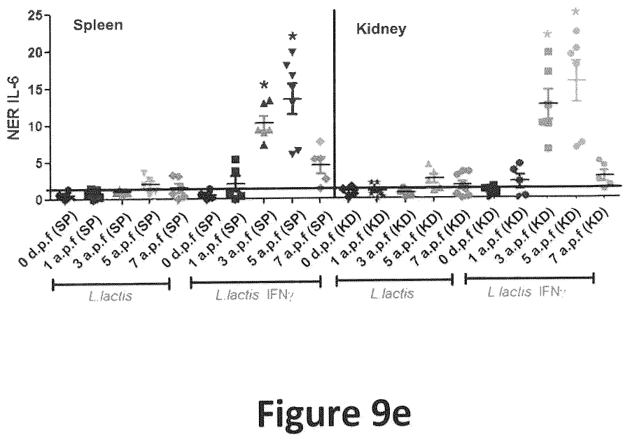

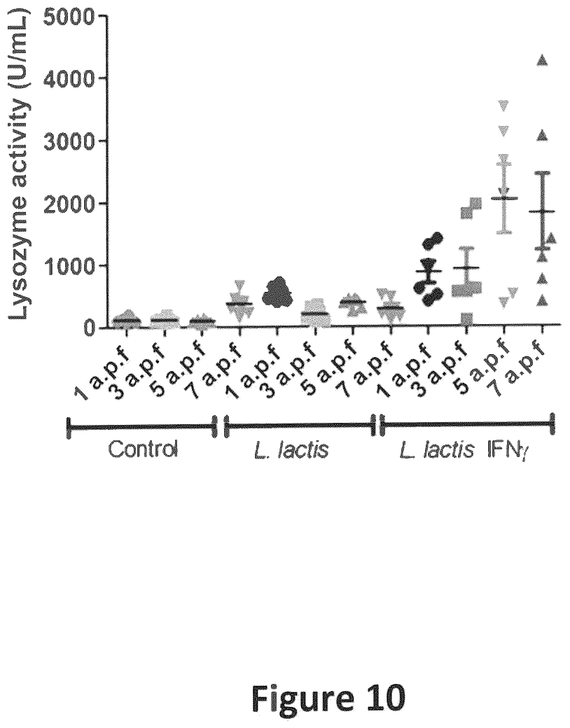

To asses the in vivo effect, transformed bacteria was administrated with feed to healthy rainbow trout specimens, then these were analyzed during and after feeding. Two paramenters were studied from spleen and kidney: a) transcripts related to immune respone of IFNg from spleen and kidney during probiotic feeding (FIG. 7) and after probiotic feeding (FIG. 9A to E) and b) lysozyme enzymatic activity from serun during (FIG. 8) and after probiotic feeding (FIG. 10). When observing transcript changes it is possible to see an increase of IL-12, STAT1 and gamma IP10, the first is involved in cellular response triggered by IFNg increase and the second two are related to the downstream response of IFNg. When studying the effect of the probiotic after feeding, no significant increase can be osberved in the transcripts related to type II IFN, but the anti-immflamatory cytokine TGF-b is stimulated and IL-6 increases considerably from the fifth day after feeding, effect that correlates to what is observed in cell culture. When quantifying lysozyme's enzymatic activity no increases are seen during feeding, however, a strong increase in seen from the fifth day after feeding as observed for IL-6 by qPCR, which suggested that the cytokine could be involved in the leveles of lysozyme in serum.

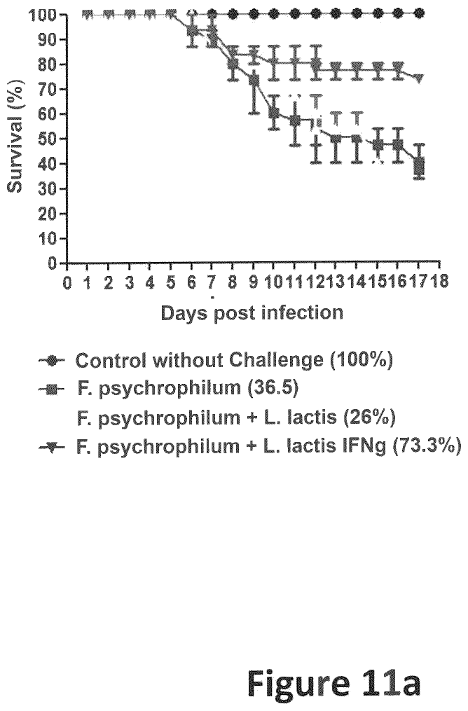

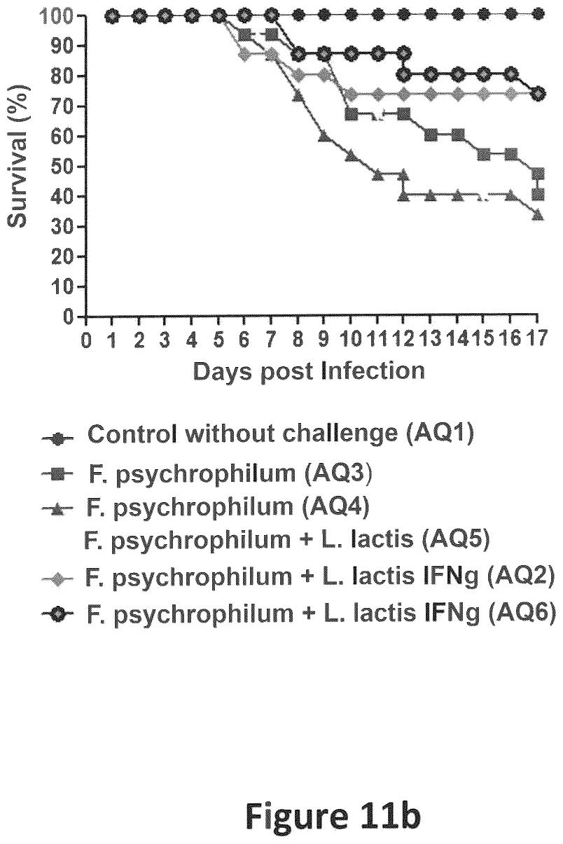

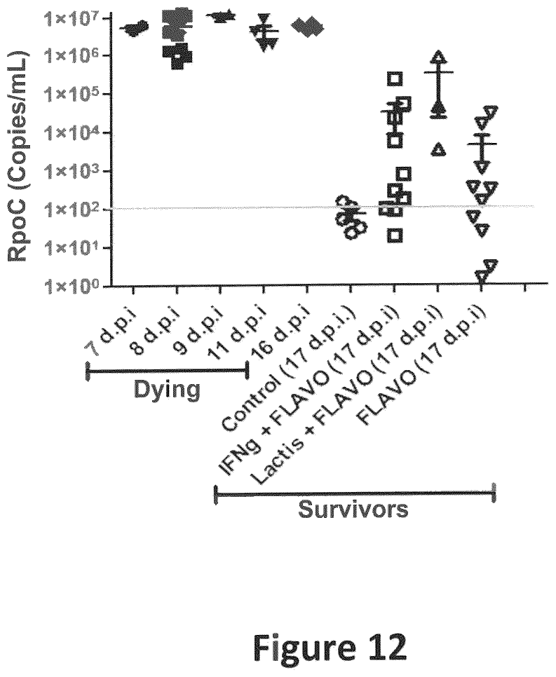

Given that estimulation of type II IFN related genes was observed, the protective effect of the probiotic was assessed, for this an experiment was set up where rainbow trout were challenged with Flavobacterium psychrophillum (FIG. 11). Fish fed with L. lactis-IFNγ (Treatment a)) presented a survival percentage higher than 70%, effect that was not seen in fish fed with L. lactis with an empty vector which only reached 35% survival (Treatment b). Fish that did not recieve probiotic treatment presented lower survival (25%) (Treament c)). When comparing bacterial load of fish that died during the experiment and surviving fish, a decline of at least 2 orders of magnitud in the number of bacterial RpoS gene copies can be observed (FIG. 12). Therefore, the heterologous protein produced by L. lactis would be protecting fish from infection of F. psychrophilum.

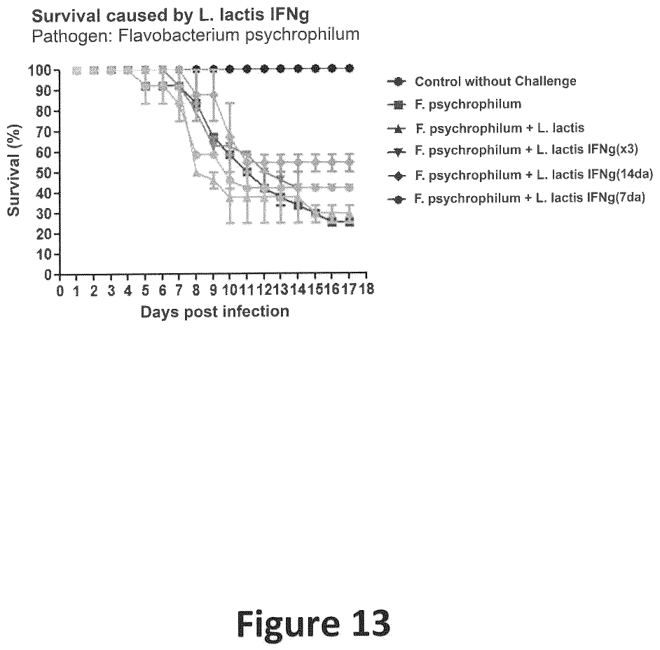

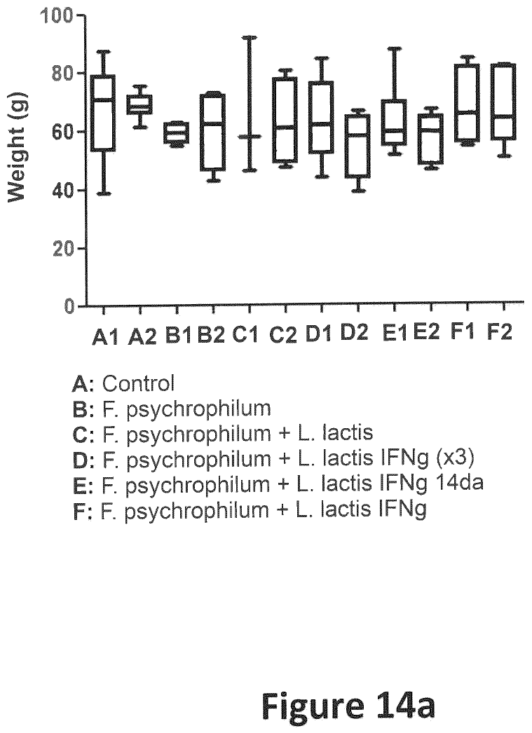

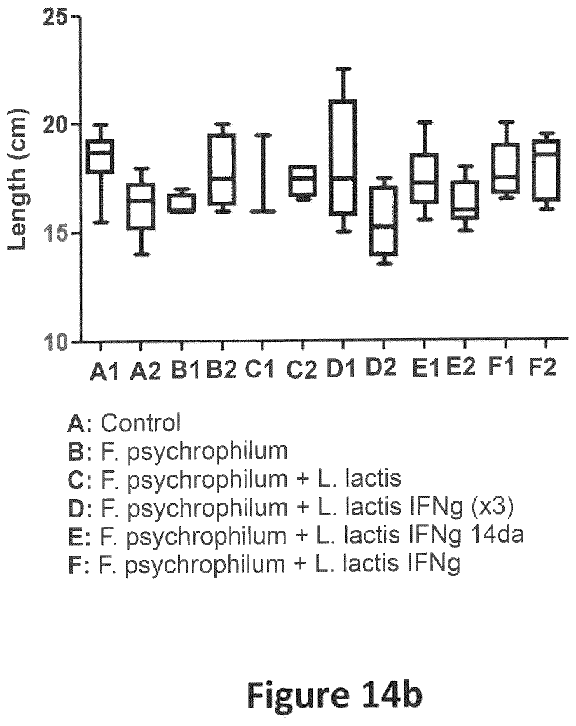

When administrating L. lactis-IFNγ for a longer period of time (13 days instead of 7), it was observed higher survival (54%) than in fish fed for 7 days (42%), the same effect was seen in fish fed with a dosage 3 times higher than the original one (42%), which indicates that prolonging feeding gives a better result (FIG. 13). Fish fed only with L. lactis (Treatment d)) presented a lower survival percentage (29%), very close to the 25% observed in fish fed with commercial feed (Treatment e)), behaviour also observed in the previous assay. The lower protection across different treatments could be due to the higher weight of fish (30-40 g versus 15-20 g), suggesting that the probiotic presents better properties in early stages of rainbow trout. When assessing weight or length of the surviving fish at the end of the experiment no significant differences were found (FIG. 14), suggestion that the probiotic does not alter the physical state of fish.

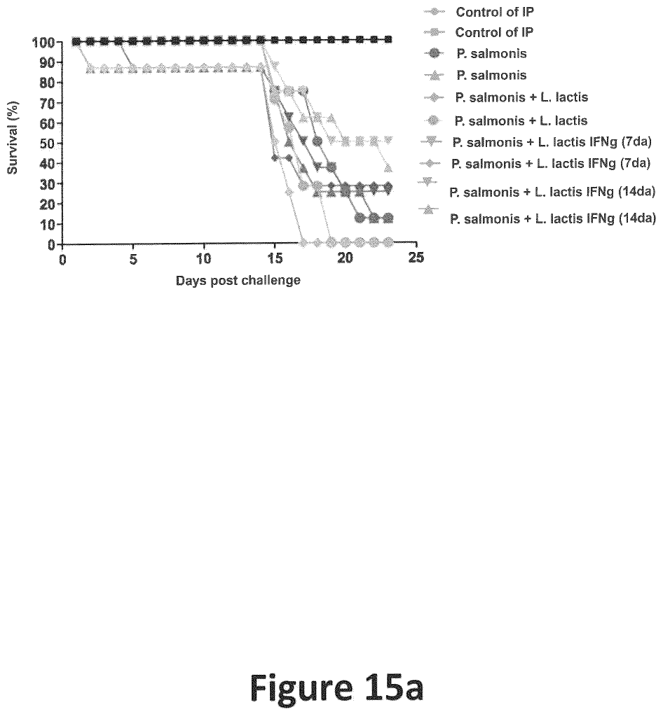

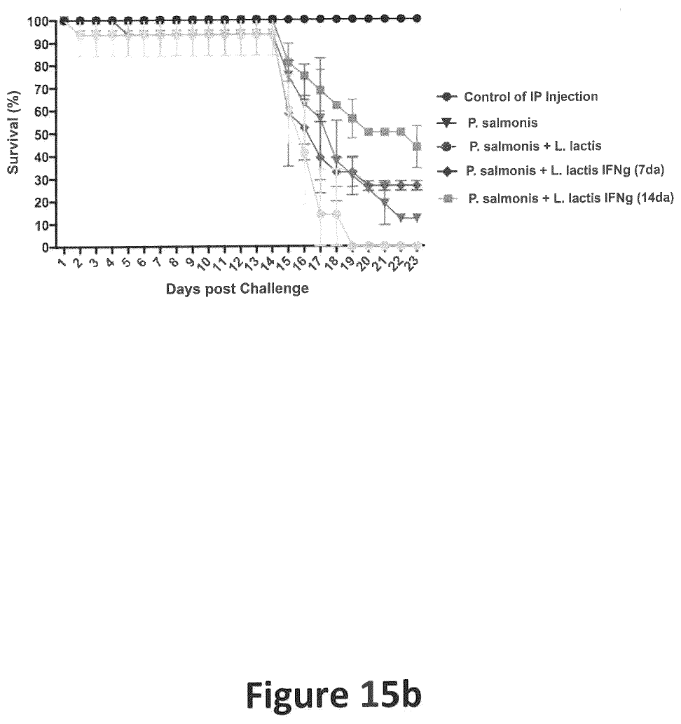

The potential use of the probiotic in Salmo salar challenged with Pisciricketssia salmonis was studied, fish fed with L. lactis-IFNg for 7 days (Treatment a: 28% survival) present a slight increase in protection compared to fish treated with the probiotic for 14 days (treatment b: 25% survival) suggesting that the protective effect against P. salmonis is given when feeding fish for 7 days and then it is maintained constant. On the other hand, fish fed with L. lactiswith an empty vector (treatment c: 0% survival) presented total mortality, however, fish fed with L. lactis with the plasmid without insert/commercial feed showed significant disparity in their mortality (Treatment d: 12% and 50% each replica). Therefore, L. lactis-IFNγ can contribute to the survival of fish infected with P. salmonis (FIG. 15).

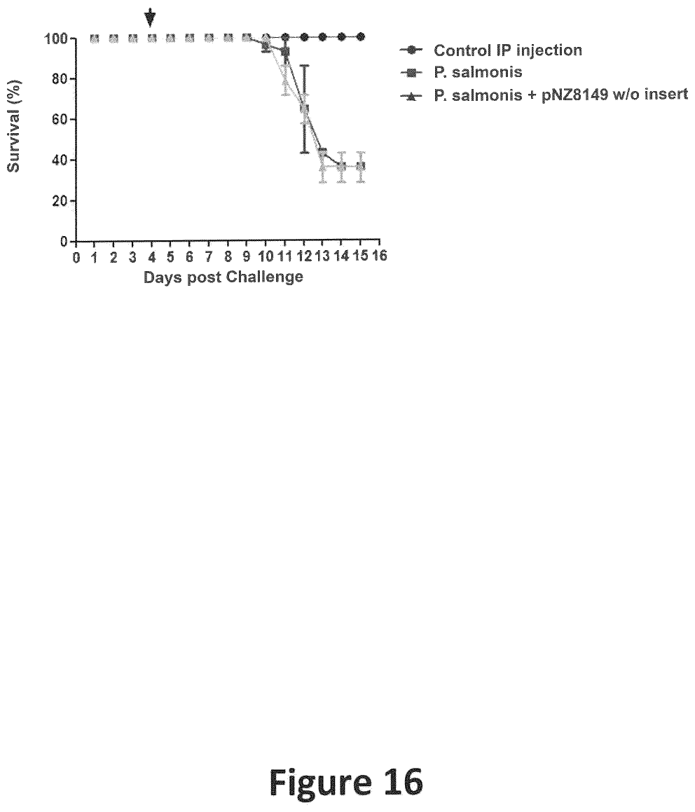

To determine if L. lactis pNZ8149 without insert has an effect on survival of fish challenged with P. salmonis, the experiment was repeated with Salmo salar specimens (average weight: 50 g) infected and fed with probiotic (FIG. 16), observing that their registered mortality was the same than for infected fish fed with commercial feed, which indicates that the bacteria per se does not confer protection.

| Sequences | |
|---|---|
| <110> | Consorcio Tecnológico de Sociedad Acuícola S.A. y Universidad de Santiago |
| <120> | Lactococcus Lactis bacteria transformed, RGM2416, producing Salmo salar |
| | interferon gamma (IFNg), feed an composition that comprises it, to immunostimulate |
| | aquatic species and prevent infection of F. psychrophilium, P. salmonis or both and |
| | methos to obtain it. |
| <160> | 5 |
| <210> | 1 |
| <211> | 779 |
| <212> | ADN |
| <400> | 1 |

| | |
|---|---|
| ccatggtata gatctaatta atctataaac catatccctc tttggaatca aaatttatta | 60 |
| tctactcctt tgtagatatg ttataataca agtatcaatg atctgggaga ccacaacggt | 120 |
| ttcccactag aaataatttt gtttaacttt agaaaggaga tatacgcatg aaaaaaaaga | 180 |
| ttatctcagc tattttaatg tctacagtga tactttctgc tgcagccccg ttgtcaggtg | 240 |
| tttacgctgc tcaatataca tcaattaata tgaaatcaaa tattgataaa cttaaagtac | 300 |
| attataaaat tagtaaagat caattgttta atggaaaacc agtttttcct aaagatacat | 360 |
| ttgaagattc agaacgtaga gtttggatgt ctgttgtatt agatgtatat cgttcaattt | 420 |
| ttaatcaaat gcttaatcaa acaggtgatc aagaagtacg tgaaagatta gatcaagtta | 480 |
| aaggaaaagt acaagaaact caaaaacatt attttcttaa acgaattcca gaattgagaa | 540 |
| cacatttgca aaatctttgg gctattgaaa ctagtaatac aactgttcaa ggaaaagcat | 600 |
| tgtcagaatt tattactatt tatgaaaaag cttctaaatt agcacttaaa attcatttaa | 660 |
| agaaagataa tcgacgtaaa agacgacaag cacaacgatt gaaaagtagt attatgggag | 720 |
| gtggacatca tcatcatcat cattaaaaaa aagtcttaaa ataataaaaa tagtctaga | |

| | |
|---|---|
| <210> | 2 |
| <211> | 161 |
| <212> | ADN |
| <400> | 2 |

| | |
|---|---|
| tatagatcta attaatctat aaaccatatc cctctttgga atcaaaattt attatctact | 60 |
| cctttgtaga tatgttataa tacaagtatc aatgatctgg gagaccacaa cggtttccca | 120 |
| ctagaaataa ttttgtttaa ctttagaaag gagatatacg c | |

| | |
|---|---|
| <210> | 3 |
| <211> | 81 |
| <212> | ADN |
| <400> | 3 |

| | |
|---|---|
| atgaaaaaaa agattatctc agctatttta atgtctacag tgatactttc tgctgcagcc | 60 |
| ccgttgtcag gtgtttacgc t | |

| | |
|---|---|
| <210> | 4 |
| <211> | 468 |
| <212> | ADN |
| <400> | 4 |

| | |
|---|---|
| gctcaatata catcaattaa tatgaaatca aatattgata aacttaaagt acattataaa | 60 |
| attagtaaag atcaattgtt taatggaaaa ccagtttttc ctaaagatac atttgaagat | 120 |
| tcagaacgta gagtttggat gtctgttgta ttagatgtat atcgttcaat ttttaatcaa | 180 |
| atgcttaatc aaacaggtga tcaagaagta cgtgaaagat tagatcaagt taaaggaaaa | 240 |
| gtacaagaaa ctcaaaaaca ttattttctt aaacgaattc cagaattgag aacacatttg | 300 |
| caaaatcttt gggctattga aactagtaat acaactgttc aaggaaaagc attgtcagaa | 360 |
| tttattacta tttatgaaaa agcttctaaa ttagcactta aaattcattt aaagaaagat | 420 |
| aatcgacgta aaagacgaca agcacaacga ttgaaaagta gtattatg | |

| | |
|---|---|
| <210> | 5 |
| <211> | 24 |
| <212> | ADN |
| <400> | 5 |

ggaggtggac atcatcatca tcat

1. A method of preparing transformed L. lactis bacteria comprising steps of:
a) digesting with restriction enzymes a plasmid that contains the reading frame of Salmo salar IFNγ codon optimized for Lactococcus lactis,
b) purifying the digestion product, corresponding to the IFNγ gene with sticky ends for restriction enzymes;
c) meanwhile, digesting the plasmid NZ8149 with enzymes;
d) purifying the linearized plasmid from an agarose gel;
e) ligating both purified products with ligase;
f) dialyzing the ligation product; and
g) transforming electrocompetent L. lactis bacteria with the ligated plasmid.

2. The method according to claim 1, wherein the enzymes of step a) are restriction enzymes NcoI and XbaI.

3. The method according to claim 1, wherein the enzymes of step c) are restriction enzymes NcoI and XbaI.

4. The method according to claims 1, wherein the electrocompetent bacteria of step g) belongs to the strain L. lactis NZ3900.

5. The method according to claim 1, wherein the enzymes of step a) and step c) are the same restriction enzymes.

6. The method according to claim 1, wherein the enzymes of step a) and step c) are the different restriction enzymes.

7. The method according to claim 1, wherein said plasmid to transform L. lactis bacteria to produce interferon gamma (IFNγ) comprising a vector pNZ8149 and a sequence P1-Usp45-IFNγ-GGG-6xHIS; wherein P1 is L. lactis constitutive expression promoter; Usp45 is a secretion signal; IFNγ is Salmo salar interferon gamma (IFNγ) mature mRNA coding sequence; GGG is a sequence coding for three glycines and 6xHis is a sequence coding for 6 terminal histidines.

8. A method of preparing transformed L. lactis bacteria comprising steps of:
a) digesting with restriction enzymes a plasmid, wherein said plasmid to transform L. lactis bacteria to produce interferon gamma (IFNγ) comprising a vector pNZ8149 and a sequence P1-Usp45-IFNγ-GGG-6xHIS; wherein P1 is L. lactis constitutive expression promoter; Usp45 is a secretion signal; IFNγ is Salmo salar interferon gamma (IFNγ) mature mRNA coding sequence; GGG is a sequence coding for three glycines and 6xHis is a sequence coding for 6 terminal histidines.
b) purifying the digestion product, corresponding to the IFNγ gene with sticky ends for restriction enzymes;
c) meanwhile, digesting the plasmid NZ8149 with enzymes;
d) purifying the linearized plasmid from an agarose gel;
e) ligating both purified products with ligase;
f) dialyzing the ligation product; and
g) transforming electrocompetent L. lactis bacteria with the ligated plasmid.

9. The method of claim 8, wherein the plasmid contains the reading frame of Salmo salar IFNγ codon optimized for Lactococcus lactis.

10. The method according to claim 1, wherein the enzymes of step a) and step c) are the same restriction enzymes.


SEQUENCE LISTING (27 sequences)

SEQ ID NO: 1 - DNA, 779 bp, synthetic construct
  misc_feature 1..779: note = gene encoding to Salmo Salar immunostimulating protein
  source 1..779: mol_type = other DNA
ccatggtatagatctaattaatctataaaccatatccctctttggaatcaaaatttattatctactcctttgtagatatgttataatacaagtatcaatgatctgggagaccacaacggtttcccactagaaataattttgtttaactttagaaaggagatatacgcatgaaaaaaaagattatctcagctattttaatgtctacagtgatactttctgctgcagccccgttgtcaggtgtttacgctgctcaatatacatcaattaatatgaaatcaaatattgataaacttaaagtacattataaaattagtaaagatcaattgtttaatggaaaaccagtttttcctaaagatacatttgaagattcagaacgtagagtttggatgtctgttgtattagatgtatatcgttcaatttttaatcaaatgcttaatcaaacaggtgatcaagaagtacgtgaaagattagatcaagttaaaggaaaagtacaagaaactcaaaaacattattttcttaaacgaattccagaattgagaacacatttgcaaaatctttgggctattgaaactagtaatacaactgttcaaggaaaagcattgtcagaatttattactatttatgaaaaagcttctaaattagcacttaaaattcatttaaagaaagataatcgacgtaaaagacgacaagcacaacgattgaaaagtagtattatgggaggtggacatcatcatcatcatcattaaaaaaaagtcttaaaataataaaaatagtctaga

SEQ ID NO: 2 - DNA, 161 bp, synthetic construct
  misc_feature 1..161: note = Primer expressing IFNg
  source 1..161: mol_type = other DNA
tatagatctaattaatctataaaccatatccctctttggaatcaaaatttattatctactcctttgtagatatgttataatacaagtatcaatgatctgggagaccacaacggtttcccactagaaataattttgtttaactttagaaaggagatatacgc

SEQ ID NO: 3 - DNA, 81 bp, synthetic construct
  misc_feature 1..81: note = secreting IFNg
  source 1..81: mol_type = other DNA
atgaaaaaaaagattatctcagctattttaatgtctacagtgatactttctgctgcagccccgttgtcaggtgtttacgct

SEQ ID NO: 4 - DNA, 468 bp, synthetic construct
  misc_feature 1..468: note = IFNg precursor
  source 1..468: mol_type = other DNA
gctcaatatacatcaattaatatgaaatcaaatattgataaacttaaagtacattataaaattagtaaagatcaattgtttaatggaaaaccagtttttcctaaagatacatttgaagattcagaacgtagagtttggatgtctgttgtattagatgtatatcgttcaatttttaatcaaatgcttaatcaaacaggtgatcaagaagtacgtgaaagattagatcaagttaaaggaaaagtacaagaaactcaaaaacattattttcttaaacgaattccagaattgagaacacatttgcaaaatctttgggctattgaaactagtaatacaactgttcaaggaaaagcattgtcagaatttattactatttatgaaaaagcttctaaattagcacttaaaattcatttaaagaaagataatcgacgtaaaagacgacaagcacaacgattgaaaagtagtattatg

SEQ ID NO: 5 - DNA, 25 bp, synthetic construct
  misc_feature 1..25: note = encoding an amino acid terminal sequence
  source 1..25: mol_type = other DNA
ggaggtgggacatcatcatcatcat

SEQ ID NO: 6 - DNA, 20 bp, synthetic construct
  misc_feature 1..20: note = FW primer IFNg salmon
  source 1..20: mol_type = other DNA
ccgtacaccgattgaggact

SEQ ID NO: 7 - DNA, 21 bp, synthetic construct
  misc_feature 1..21: note = RV primer IFNg salmon
  source 1..21: mol_type = other DNA
gccggcattactccatcctaa

SEQ ID NO: 8 - DNA, 20 bp, synthetic construct
  misc_feature 1..20: note = FW primer IL1b salmon
  source 1..20: mol_type = other DNA
ccccattgagactaaagcca

SEQ ID NO: 9 - DNA, 20 bp, synthetic construct
  misc_feature 1..20: note = RV primer IL1b salmon
  source 1..20: mol_type = other DNA
gcaacctcctctaggtgcag

SEQ ID NO: 10 - DNA, 20 bp, synthetic construct
  misc_feature 1..20: note = FW primer TGF-B salmon
  source 1..20: mol_type = other DNA
agctctcggaagaaacgaca

SEQ ID NO: 11 - DNA, 20 bp, synthetic construct
  misc_feature 1..20: note = RV primer TGF-B salmon
  source 1..20: mol_type = other DNA
agtagccagtgggttcatgg

SEQ ID NO: 12 - DNA, 22 bp, synthetic construct
  misc_feature 1..22: note = FW primer gamma IP10 salmon
  source 1..22: mol_type = other DNA
gtgtctgaatccagaggctcca

SEQ ID NO: 13 - DNA, 22 bp, synthetic construct
  misc_feature 1..22: note = RV primer gamma IP10 salmon
  source 1..22: mol_type = other DNA
tctcatggtgctctctgttcca

SEQ ID NO: 14 - DNA, 23 bp, synthetic construct
  misc_feature 1..23: note = FW primer IFNg Lactis-IFNg
  source 1..23: mol_type = other DNA
cacatttgcaaaatctttgggct

SEQ ID NO: 15 - DNA, 21 bp, synthetic construct
  misc_feature 1..21: note = RV primer IFNg Lactis-IFNg
  source 1..21: mol_type = other DNA
caatcgttgtgcttgtcgtct

SEQ ID NO: 16 - DNA, 21 bp, synthetic construct
  misc_feature 1..21: note = FW primer IL-6 salmon
  source 1..21: mol_type = other DNA
ccttgcggaaccaacagtttg

SEQ ID NO: 17 - DNA, 22 bp, synthetic construct
  misc_feature 1..22: note = RV primer IL-6 salmon
  source 1..22: mol_type = other DNA
cctcagcaaccttcatctggtc

SEQ ID NO: 18 - DNA, 23 bp, synthetic construct
  misc_feature 1..23: note = FW primer IL-12 salmon
  source 1..23: mol_type = other DNA
tgacgctttttctcaccggttgt

SEQ ID NO: 19 - DNA, 22 bp, synthetic construct
  misc_feature 1..22: note = RV primer IL-12 salmon
  source 1..22: mol_type = other DNA
acgctttgcagcatgagcttga

SEQ ID NO: 20 - DNA, 20 bp, synthetic construct
  misc_feature 1..20: note = FW primer eF1a salmon
  source 1..20: mol_type = other DNA
gggtgagtttgaggctggta

SEQ ID NO: 21 - DNA, 20 bp, synthetic construct
  misc_feature 1..20: note = RV primer eF1a salmon
  source 1..20: mol_type = other DNA
ttctggatctcctcaaaccg

SEQ ID NO: 22 - DNA, 30 bp, synthetic construct
  misc_feature 1..30: note = FW primer RpoS F. pyschrophilum
  source 1..30: mol_type = other DNA
gaagatggagaaggtaatttagttgatatt

SEQ ID NO: 23 - DNA, 30 bp, synthetic construct
  misc_feature 1..30: note = RV primer RpoS F. pyschrophilum
  source 1..30: mol_type = other DNA
caaataacatctcctttttctacaacttga

SEQ ID NO: 24 - DNA, 24 bp, synthetic construct
  misc_feature 1..24: note = FW primer STAT1
  source 1..24: mol_type = other DNA
gaccagcgaacccaagaacctgaa

SEQ ID NO: 25 - DNA, 23 bp, synthetic construct
  misc_feature 1..23: note = RV primer STAT1
  source 1..23: mol_type = other DNA
cacaaagcccaggatgcaaccat

SEQ ID NO: 26 - DNA, 19 bp, synthetic construct
  misc_feature 1..19: note = FW primer rDNA 16S P. salmonis
  source 1..19: mol_type = other DNA
agggagactgccggtgata

SEQ ID NO: 27 - DNA, 19 bp, synthetic construct
  misc_feature 1..19: note = RV primer rDNA 16S P. salmonis
  source 1..19: mol_type = other DNA
actacgaggcgctttctca